(19) United States
(12) Patent Application Publication
(10) Pub. No.: US 2002/0151929 A1
(43) Pub. Date: Oct. 17, 2002

(54) Venous thromboembolism preventing apparatus

(75) Inventors: Masami Goto, Komaki-shi (JP); Tsuneo Nakagawa, Komaki-shi (JP); Tomohiro Nunome, Komaki-shi (JP)

Correspondence Address: OLIFF & BERRIDGE, PLC, P.O. BOX 19928, ALEXANDRIA, VA, 22320, US

(73) Assignee: COLIN CORPORATION, Komaki-shi (JP)

(21) Appl. No.: 09/934,758

(22) Filed: Aug. 23, 2001

(30) Foreign Application Priority Data
Apr. 11, 2001 (JP) 2001-112133

(51) Int. Cl. A61B 17/00

(52) U.S. Cl. 606/202

(57) ABSTRACT

An apparatus for preventing a venous thromboembolism of a living subject, including an inflatable cuff which is adapted to be wound around a body portion of the subject and applies a pressing pressure to the body portion so as to press the body portion and thereby prevent the venous thromboembolism; a blood-pressure-relating-information obtaining device which obtains blood-pressure-relating information which changes corresponding to blood pressure of the subject; a pressing-pressure determining device for determining the pressing pressure of the inflatable cuff, based on the blood-pressure-relating information obtained by the blood-pressure-relating-information obtaining device, according to a prescribed relationship between pressing pressure and blood-pressure-relating information, in which the pressing pressure increases as the blood pressure corresponding to the blood-pressure-relating information increases; and a pressing device which operates the inflatable cuff to apply the pressing pressure determined by the pressing-pressure determining device, to the body portion of the subject so as to press the body portion and thereby prevent the venous thromboembolism.

### BACKGROUND OF THE INVENTION

1. Field of the Invention

The present invention relates to an apparatus for preventing a venous thromboembolism of a living subject by pressing a body portion of the subject. The present invention also relates to an apparatus for preventing a venous thromboembolism of a living subject by pressing an inferior limb of the subject starting with a distal-side portion of the limb and thereby promoting flow of blood in veins of the limb.

2. Related Art Statement

Before or after a surgical operation, a patient needs to rest on a bed without moving his or her superior or inferior limbs for a long time. If the patient does not move his or her superior or inferior limbs for a long time, then blood tends to stay in large venous sinuses that are present in muscles and are free of valves. Normally, the contractions of the muscles send the blood staying in the veins, back to the central side, i.e., the heart. In a special case, however, in which the patient's muscle force is not so strong, the blood cannot flow so fast and accordingly tends to stay in the veins. Thus, the patient may suffer thrombi (or phlebothrombosis). The phlebothrombosis tends to occur to calves of inferior limbs.

If thrombi occur to veins of a patient, he or she feels pains, or suffers swelling or pressure pains, around the veins. In addition, if the thrombi (or clots) grow up and then flow through the veins to the lungs, they clog up the pulmonary arteries, thereby producing pectoralgia, respiration distress, or expectoration (i.e., venous thromboembolism).

There has been known a venous thromboembolism preventing method in which superior or inferior limbs are intermittently pressed to promote flow of blood in veins, and there has been proposed a venous thromboembolism preventing apparatus which can carry out this method. This apparatus includes an inflatable cuff which is adapted to be wound around a body portion, such as an inferior limb, of a living subject and which is inflatable to press the body portion and thereby promote flow of blood in veins. Since promoting flow of blood in veins prevents phlebothrombosis, it also prevents venous thromboembolism.

However, in the above-indicated conventional venous thromboembolism preventing apparatus, the cuff applies a prescribed pressing pressure to a body portion of a patient. Thus, in some cases, the pressing pressure is too low to sufficiently prevent phlebothrombosis, or is too high and causes the patient to feel unnecessary pains.

In addition, there has been proposed another venous thromboembolism preventing apparatus which includes a plurality of pressing bands which are adapted to be wound around respective portions of an inferior limb of a living subject such that the pressing bands are located on both sides of the calf of the inferior limb, and a pressing force of each of which is changeable to press the inferior limb, starting with a distal-side portion of the limb, and thereby promote flow of blood in veins of the limb so as to prevent venous thromboembolism.

However, the above-indicated second venous thromboembolism preventing apparatus periodically presses the inferior limb at a prescribed pressing period. Thus, the conventional apparatus presses the inferior limb even in a state in which there is no concern about venous thromboembolism, and causes a patient to feel unnecessary pains. In particular, if an inferior limb of a patient who is sleeping is pressed at a prescribed period, he or she cannot deeply sleep.

### SUMMARY OF THE INVENTION

It is therefore an object of the present invention to provide a venous thromboembolism preventing apparatus which applies an appropriate pressing pressure to a body portion of a living subject so as to prevent venous thromboembolism.

To this end, inventors have carried out extensive studies and have found that when an inflatable cuff is used to press a body portion of a patient to promote flow of blood, it is needed to apply a higher pressing pressure to the body portion of the patient who has a higher blood pressure. In other words, a lower pressing pressure can be applied to a body portion of a patient who has a lower blood pressure, without causing the patient to feel unnecessary pains. The present invention has been developed based on this finding.

The above object has been achieved by the present invention. According to a first aspect of the present invention, there is provided an apparatus for preventing a venous thromboembolism of a living subject, comprising an inflatable cuff which is adapted to be wound around a body portion of the subject and applies a pressing pressure to the body portion so as to press the body portion and thereby prevent the venous thromboembolism; a blood-pressure-relating-information obtaining device which obtains blood-pressure-relating information which changes corresponding to blood pressure of the subject; a pressing-pressure determining means for determining the pressing pressure of the inflatable cuff, based on the blood-pressure-relating information obtained by the blood-pressure-relating-information obtaining device, according to a prescribed relationship between pressing pressure and blood-pressure-relating information, in which the pressing pressure increases as the blood pressure corresponding to the blood-pressure-relating information increases; and a pressing device which operates the inflatable cuff to apply the pressing pressure determined by the pressing-pressure determining means, to the body portion of the subject so as to press the body portion and thereby prevent the venous thromboembolism.

In this venous thromboembolism preventing apparatus, a higher pressing pressure is determined, by the pressing-pressure determining means, based on a higher blood pressure corresponding to a piece of blood-pressure-relating information actually obtained. That is, a lower pressing pressure is determined, by the pressing-pressure determining means, based on a lower blood pressure corresponding to a piece of blood-pressure-relating information actually obtained. Since the pressing device operates the inflatable cuff to press, with the thus determined pressing pressure, the body portion of the living subject, the body portion is pressed with the appropriate pressing pressure.

It is another object of the present invention to provide a venous thromboembolism preventing apparatus which presses an inferior limb of a living subject only a minimized number of times.

To this end, inventors have carried out extensive studies and have found that if an inferior limb is pressed only when phlebostasis as a sign of thrombi occurs to cnemial veins, the number of pressing times can be minimized. The present invention has been developed based on this finding.

The above second object has been achieved by the present invention. According to a second aspect of the present invention, there is provided an apparatus for preventing a venous thromboembolism of a living subject, comprising at least two pressing bands which are adapted to be wound around a distal-side portion and a proximal-side portion of an inferior limb of the subject that are located on a distal side and a proximal side of a calf of the inferior limb, respectively, and which apply respective changeable pressing forces to the distal-side portion and the proximal-side portion, such that a distal-side one of the pressing bands earlier starts applying a corresponding one of the changeable pressing forces to the distal-side portion than the other, proximal-side pressing band starts applying the other changeable pressing force to the proximal-side portion, so as to promote flow of blood in veins of the inferior limb and thereby prevent the venous thromboembolism; a phlebostasis-relating-information obtaining device which obtains, from at least physical information obtained from a distal-side portion of the inferior limb that is located on a distal side of a knee of the subject, phlebostasis-relating information which changes in relation with phlebostasis of the veins of the inferior limb; a phlebostasis judging means for judging that the veins of the inferior limb have phlebostasis, when the phlebostasis-relating information obtained by the phlebostasis-relating-information obtaining device does not fall within a reference range; and a blood-flow promoting means for operating, when the phlebostasis judging means judges that the veins of the inferior limb have phlebostasis, the distal-side and proximal-side pressing bands to apply the respective changeable pressing forces to the distal-side and proximal-side portions of the inferior limb, such that the distal-side pressing band earlier starts applying the one changeable pressing force to the distal-side portion of the inferior limb than the proximal-side pressing band starts applying the other changeable pressing force to the proximal-side portion of the inferior limb, so as to promote the flow of blood in the veins of the inferior limb and thereby prevent the venous thromboembolism.

In this venous thromboembolism preventing apparatus, the phlebostasis judging means judges that the veins of the inferior limb have phlebostasis, based on whether the phlebostasis-relating information obtained by the phlebostasis-relating-information obtaining device does not fall within the reference range and, only when the veins of the inferior limb is judged to have phlebostasis, the blood-flow promoting means operates the plurality of pressing bands to press the inferior limb and thereby promote the flow of blood in the veins. Thus, the number of pressing times can be minimized.

According to a preferred feature of the second aspect of the present invention, the presenting apparatus further comprises a superior-limb-blood-pressure measuring device which iteratively measures a superior-limb blood pressure of a superior limb of the subject; and an inferior-limb-blood-pressure measuring device which iteratively measures, as the physical information, an inferior-limb blood pressure of the distal-side portion of the inferior limb that is located on the distal side of the knee, and the phlebostasis-relating-information obtaining device iteratively obtains a piece of phlebostasis-relating information based on each of the superior-limb blood pressure values iteratively measured by the superior-limb-blood-pressure measuring device and each of the inferior-limb blood pressure values iteratively measured by the inferior-limb-blood-pressure measuring device.

When phlebostasis occurs to the veins of the inferior limb, the inferior-limb blood pressure increases. On the other hand, the superior-limb blood pressure is not influenced by the phlebostasis of the veins of the inferior limb. Therefore, the phlebostasis-relating information can be obtained based on the superior-limb blood pressure and the inferior-limb blood pressure.

According to another feature of the second aspect of the present invention, the presenting apparatus further comprises an inferior-limb-pulse-wave detecting device which detects, as the physical information, an inferior-limb pulse wave from the distal-side portion of the inferior limb that is located on the distal side of the knee; and a pulse-wave-amplitude determining means for determining an amplitude of each of heartbeat-synchronous pulses of the inferior-limb pulse wave detected by the inferior-limb-pulse-wave detecting device, and the phlebostasis-relating-information obtaining device iteratively obtains a piece of phlebostasis-relating information based on the determined amplitude of the each of the heartbeat-synchronous pulses of the inferior-limb pulse wave.

When phlebostasis occurs to the veins of the inferior limb, those veins resist the flow of blood present on the distal side of the knee of the inferior limb. In this case, the amplitude of each heartbeat-synchronous pulse of the inferior-limb pulse wave detected by the inferior-limb-pulse-wave detecting device decreases. Therefore, the phlebostasis-relating information can be obtained based on the amplitude of each heartbeat-synchronous pulse of the inferior-limb pulse wave.

According to another feature of the second aspect of the present invention, the presenting apparatus further comprises an inferior-limb-pulse-wave detecting device which detects, as the physical information, an inferior-limb pulse wave from the distal-side portion of the inferior limb that is located on the distal side of the knee; and a sharpness-degree determining means for determining a degree of sharpness of each of heartbeat-synchronous pulses of the inferior-limb pulse wave detected by the inferior-limb-pulse-wave detecting device, and the phlebostasis-relating-information obtaining device iteratively obtains a piece of phlebostasis- relating information based on the determined degree of sharpness of the each of the heartbeat-synchronous pulses of the inferior-limb pulse wave.

When phlebostasis occurs to the veins of the inferior limb, those veins resist the flow of blood present on the distal side of the knee of the inferior limb. In this case, the shape of each heartbeat-synchronous pulse of the inferior-limb pulse wave detected by the inferior-limb-pulse-wave detecting device becomes duller. Therefore, the phlebostasis-relating information can be obtained based on the sharp degree of each heartbeat-synchronous pulse of the inferior-limb pulse wave.

According to another feature of the second aspect of the present invention, the presenting apparatus further comprises a weight measuring device which supports an under-knee portion of the inferior limb of the living subject who is taking a face-up position, and which iteratively measures a weight of the under-knee portion, wherein the phlebostasis-relating-information obtaining device iteratively obtains a piece of phlebostasis-relating information based on each of the iteratively measured weights of the under-knee portion.

When phlebostasis occurs to the veins of the inferior limb, the weight of the under-knee portion of the inferior limb increases by the amount of blood staying in the veins, and accordingly the weight of the under-knee portion iteratively measured by the weight measuring device gradually increases. Therefore, the phlebostasis-relating information can be obtained based on each of the weights of the under-knee portion iteratively measured by the weight measuring device.

According to another feature of the second aspect of the present invention, the presenting apparatus further comprises a circumferential-length measuring device which iteratively measures a circumferential length of a portion of the inferior limb that is located between a knee thereof and an ankle thereof, wherein the phlebostasis-relating-information obtaining device iteratively obtains a piece of phlebostasis-relating information based on each of the iteratively measured circumferential lengths of the under-knee portion.

When phlebostasis occurs to the veins of the inferior limb, the circumferential length of the portion of the inferior limb located between its knee and its ankle increases by the amount of blood staying in the veins. Therefore, the phlebostasis-relating information can be obtained based on each of the iteratively measured circumferential lengths of any portion of the inferior portion located between its knee and its ankle.

### BRIEF DESCRIPTION OF THE DRAWINGS

The above and optional objects, features, and advantages of the present invention will be better understood by reading the following detailed description of the preferred embodiments of the invention when considered in conjunction with the accompanying drawings, in which:

FIG. 1 is a diagrammatic view for explaining a construction of a venous thromboembolism preventing apparatus to which the present invention is applied;

FIG. 2 is a block diagram for explaining essential functions of a control device of the apparatus of FIG. 1;

FIG. 3 is a graph showing a pre-stored relationship between blood-flow promoting pressure PP and diastolic blood pressure BP(DIA);

FIG. 4 is a flow chart representing a blood-flow-promoting-pressure determining routine according to which the control device of FIG. 2 operates;

FIG. 5 is a flow chart representing a blood-flow promoting routine according to which the control device of FIG. 2 operates;

FIG. 6 is a graph showing respective ankle pulse waves ML which are detected by a pulse-wave filter circuit of the apparatus of FIG. 1 when cnemial veins have phlebostasis and when cnemial veins do not have phlebostasis;

FIG. 7 is a diagrammatic view corresponding to FIG. 1, for explaining a construction of another venous thromboembolism preventing as a second embodiment of the present invention;

FIG. 8 is a block diagram corresponding to FIG. 2, for explaining essential functions of a control device of the apparatus of FIG. 7;

FIG. 9 is a flow chart representing a control program according to which the control device of FIG. 10 operates;

FIG. 10 is a flow chart representing a blood-pressure measuring routine which is carried out at Step SA4 of FIG. 9;

FIG. 11 is a flow chart representing a blood-flow-promotion control routine which is carried out at Step SA8 of FIG. 9;

FIG. 12 is a graph representing respective changes of respective pressing pressures PARDC, PFRDC of a right-femur cuff and a right-ankle cuff according to the blood-flow- promotion control routine of FIG. 11;

FIG. 13 is a diagrammatic view corresponding to FIG. 1, for explaining a construction of another venous thromboembolism preventing as a third embodiment of the present invention;

FIG. 14 is a block diagram corresponding to FIG. 2, for explaining essential functions of a control device of the apparatus of FIG. 13;

FIG. 15 is a flow chart representing a control program according to which the control device of FIG. 11 operates;

FIG. 16 is a diagrammatic view corresponding to FIG. 1, for explaining a construction of another venous thromboembolism preventing as a fourth embodiment of the present invention;

FIG. 17 is a block diagram corresponding to FIG. 2, for explaining essential functions of a control device of the apparatus of FIG. 16;

FIG. 18 is a graph showing a heartbeat-synchronous pulse of a photoelectric pulse wave detected by a photoelectric-pulse-wave sensor shown in FIG. 17;

FIG. 19 is a block diagram corresponding to FIG. 2, for explaining essential functions of a control device of another venous thromboembolism preventing as a fifth embodiment of the present invention;

FIG. 20 is a view of a circumferential-length-change measuring device which is employed in another venous thromboembolism preventing as a sixth embodiment of the present invention; and

FIG. 21 is a block diagram corresponding to FIG. 2, for explaining essential functions of a control device of the apparatus of FIG. 20.

### DETAILED DESCRIPTION OF PREFERRED EMBODIMENTS

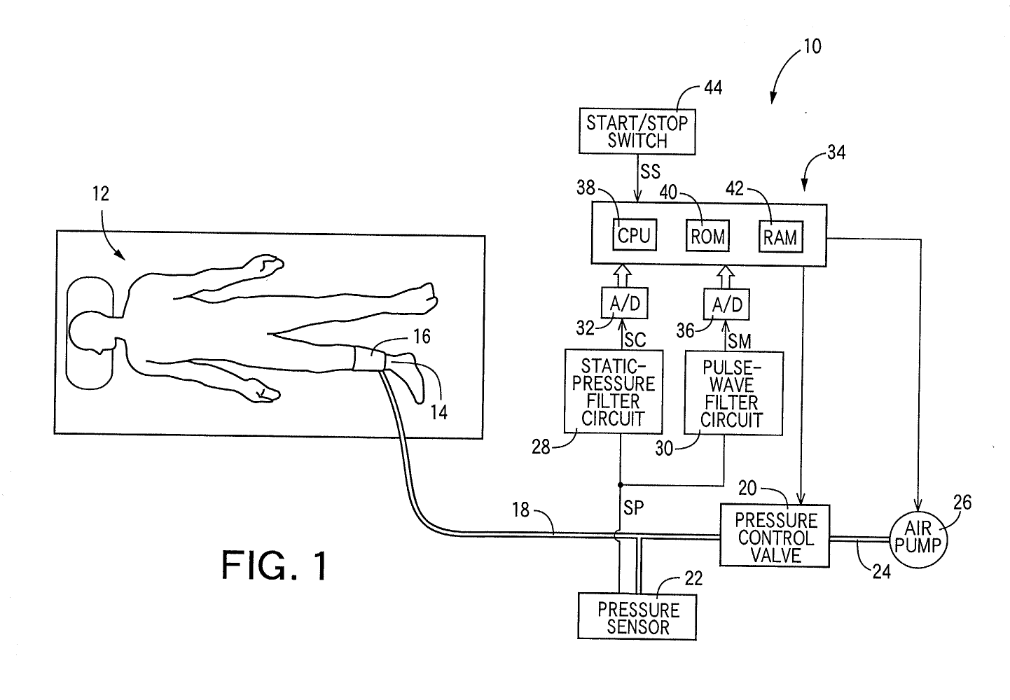

Hereinafter, there will be described an embodiment of the present invention in detail by reference to the accompanying drawings. FIG. 1 is a diagrammatic view showing a construction of a venous thromboembolism preventing apparatus 10 to which the present invention is applied.

In FIG. 1, the venous thromboembolism preventing apparatus 10 includes an inflatable cuff (i.e., a pressing band) 16 which is adapted to be wound around an ankle 14 of a patient 12 who is taking a face-up position. The cuff 16 includes a belt-like outer bag which is formed of non-stretchable cloth or polyester and a rubber bag accommodated in the outer bag and which has a width of, e.g., 12 cm.

The cuff 16 is connected to a pressure control valve 20 and a pressure sensor 22 via a piping 18. The pressure control valve 20 is connected to an air pump 26 via a piping 24.

The pressure sensor 22 detects an air pressure in the cuff 16, and supplies a pressure signal SP representing the detected pressure, to each of a static-pressure filter circuit 28 and a pulse-wave filter circuit 30. The pressure control valve 20 is selectively placed in a pressure-supply position in which the control valve 20 permits a pressurized air to be supplied from the air pump 26 to the cuff 16, a pressure-keep position in which the control valve 20 keeps the air pressure in the cuff 16, a slow-deflation position in which the control valve 20 permits the pressurized air to be slowly discharged from the cuff 16, and a quick-deflation position in which the control valve 20 permits the pressurized air to be quickly discharged from the cuff 16.

The static-pressure filter circuit 28 includes a low-pass filter and extracts, from the pressure signal SP, a static-pressure component PC contained in the signal SP, i.e., a cuff-pressure signal SC representing the static pressure PC in the cuff 16. The cuff-pressure signal SC is supplied to a control device 34 via an A/D (analog-to-digital) converter 32. The pulse-wave filter circuit 30 includes a band-pass filter and extracts, from the pressure signal SP, an oscillating component having predetermined frequencies, i.e., a pulse-wave signal SM. The pulse-wave signal SM is supplied to the control device 34 via an A/D converter 36. The pulse-wave signal SM represents a cuff pulse wave, i.e., a pressure wave which is produced from an artery of the patient in synchronism with the heartbeat of the patient and is propagated to the cuff 16.

The control device 34 is provided by a so-called microcomputer including a CPU (central processing unit) 38, a ROM (read only memory) 40, a RAM (random access memory) 42 and an I/O (input-and-output) port, not shown. The CPU 38 processes signals according to control programs pre-stored in the ROM 40 by utilizing a temporary-storage function of the RAM 42, and supplies control signals to the pressure control valve 20 and the air pump 26 through the I/O port. In addition, the CPU 38 determines, based on the cuff-pressure signal SC supplied from the static-pressure filter circuit 28 and the pulse-wave signal SM supplied from the pulse-wave filter circuit 30, a blood-pressure (BP) value of the patient and a pressing pressure PP of the cuff 16 to promote flow of blood of the patient (hereinafter, referred to as the blood-flow promoting pressure PP).

The present apparatus 10 further includes a START/STOP switch 44 which supplies, each time it is operated by an operator, a START/STOP signal SS to the control device 34 so as to alternately start and stop the apparatus 10.

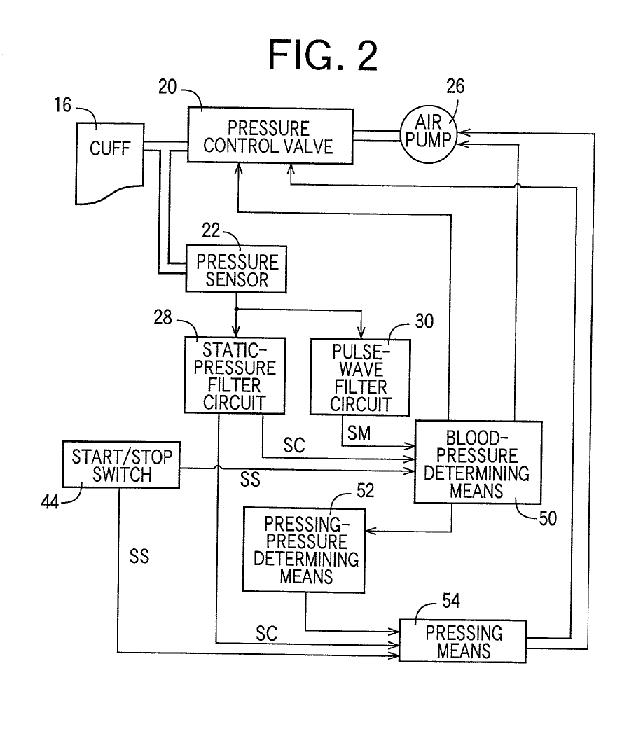

FIG. 2 is a block diagram for explaining essential functions of the control device 34. In the figure, a blood-pressure (BP) determining means 50 as a BP-relating-information obtaining means is started when the control device 34 receives a START/STOP signal SS from the START/STOP switch 44 in a state in which the present apparatus 10 is stopped. The BP determining means 50 changes the cuff pressure PC by controlling the air pump 26 and the pressure control valve 20, and determines a BP value BP of the patient based on the cuff-pressure signal SC and the pulse-wave signal SM which are obtained while the cuff pressure PC is changed. More specifically described, first, the BP determining means 50 controls the air pump 26 and the pressure control valve 20 to quickly increase the cuff pressure PC up to a prescribed target pressure P_{CM} (e.g., 180 mmHg) and then slowly decrease the cuff pressure PC at a rate of 3 mmHg/sec. Subsequently, the BP determining means 50 determines, based on the cuff-pressure signal SC continuously supplied from the static-pressure filter circuit 28 and the pulse-wave signal SM continuously supplied from the pulse-wave filter circuit 30 during the slow decreasing of the cuff pressure PC, a systolic BP value BP(SYS), a mean BP value BP(MEAN), and a diastolic BP value BP(DIA) of the ankle 14 of the patient 12, according to well-known oscillometric method. After determining the diastolic BP value BP(DIA), the BP determining means 50 quickly deflates the cuff 16.

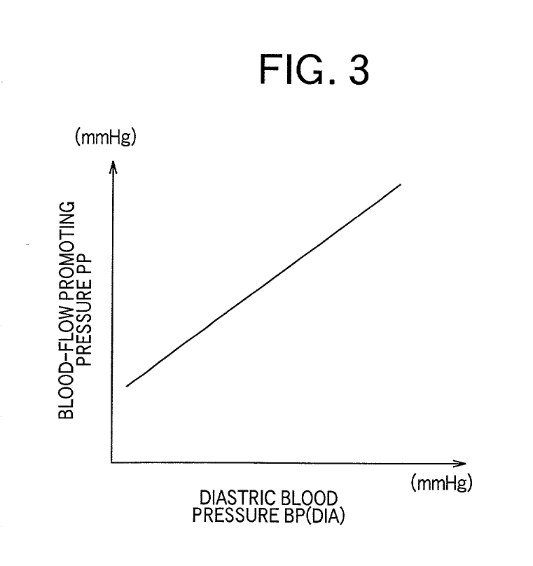

A pressing pressure determining means 52 determines, based on an actual BP value BP determined by the BP determining means 50, a blood-flow promoting pressure PP to be used by a pressing means 54, described below, according to a prescribed relationship between blood-flow promoting pressure PP and blood pressure BP. The prescribed relationship is pre-stored in the ROM 40. As described previously, when the cuff 16 is used to press an inferior limb and thereby promote flow of blood in the inferior limb, it is desirable to apply a higher blood-flow promoting pressure PP to a patient who has a higher blood-pressure value BP. Therefore, the pre-stored relationship is prescribed, based on experimental results, such that blood-flow promoting pressure PP monotonously increases as blood pressure BP increases. The sort of blood pressure BP employed in the above relationship may be any one of diastolic blood pressure BP(DIA), mean blood pressure BP(MEAN), and systolic blood pressure BP(SYS). However, diastolic blood pressure BP(DIA) is the most preferable, because thrombi occur to veins and diastolic blood pressure BP(DIA) most closely relates to venous pressure. FIG. 3 shows an example of the prescribed relationship in which diastolic blood pressure BP(DIA) is employed.

The pressing means 54 operates the cuff 16 to apply the blood-flow promoting pressure PP determined by the pressing pressure determining means 52, to the ankle 14, at a prescribed pressing period T₁. More specifically described, the pressing means 54 controls, at the pressing period T₁, the air pump 26 and the pressure control valve 20, based on the cuff-pressure signal SC supplied from the static-pressure filter circuit 28, so as to quickly increase the cuff pressure PC to the blood-flow promoting pressure PP. Then, after the cuff pressure PC is kept at the promoting pressure PP for a prescribed pressure-keep time, or immediately after the cuff pressure PC reaches the promoting pressure PP, the cuff pressure PC is quickly decreased. The pressing period T₁ is prescribed, based on experimental results, at, e.g., one hour.

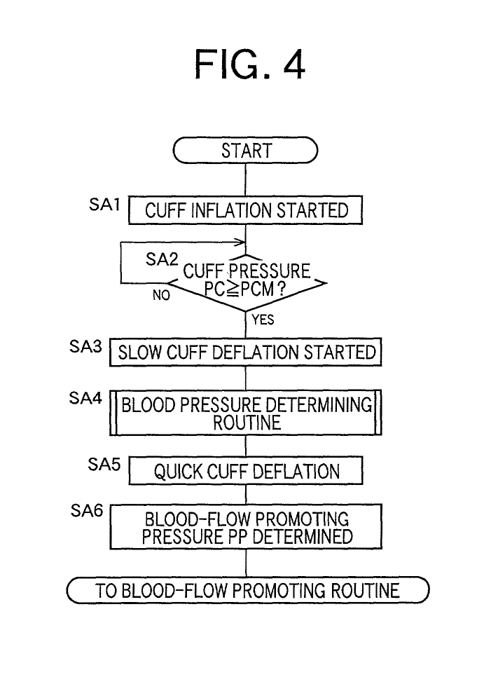

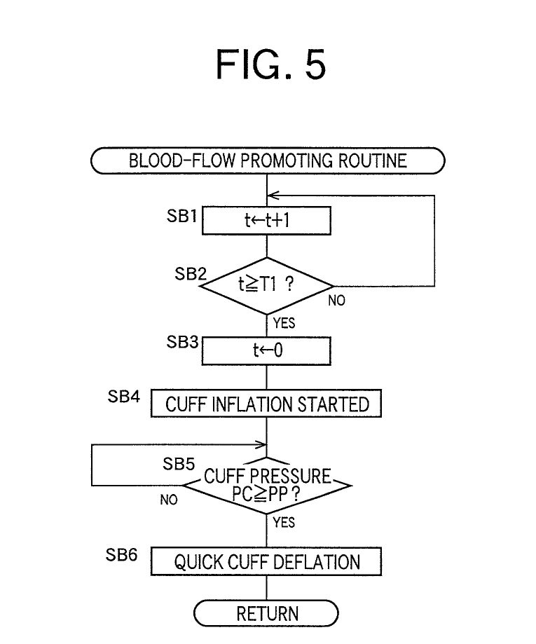

FIG. 4 and FIG. 5 show two flow charts representing a blood-flow-promoting-pressure determining routine and a blood-flow promoting routine, respectively, according to which the control device 34 is operated.

The blood-flow-promoting-pressure determining routine represented by the flow chart of FIG. 4 is carried out when the START/STOP switch 44 is operated to supply the START/STOP signal SS to the control device 34 in the state in which the present venous thromboembolism preventing apparatus 10 is stopped. First, at Step SA1 of FIG. 4 (hereinafter, “Step” is omitted, if appropriate), the pressure control valve 20 is switched to the pressure-supply position, and the air pump 26 is started, so that quick inflation of the cuff 16 is started. Then, at SA2, the control device 34 judges whether the cuff pressure PC has been increased up to the target pressure P_{CM} equal to 180 mmHg. If a negative judgment is made at SA2, SA2 is repeated while the increasing of the cuff pressure PC is continued.

Meanwhile, if a positive judgment is made at SA2, the control goes to SA3 to switch the pressure control valve 20 to the slow-deflation position, so that the air pressure in the cuff 16 is slowly decreased at the prescribed rate of 3 mmHg/sec.

At SA4, a BP determining routine is carried out. More specifically described, an amplitude of each of heartbeat-synchronous pulses of the pulse-wave signal SM continuously supplied from the pulse-wave filter circuit 30 is determined and, based on the change of the thus determined amplitudes, a systolic BP value BP(SYS), a mean BP value BP(MEAN), and a diastolic BP value BP(DIA) of the patient are determined according to a well-known oscillometric BP determining algorithm.

After the diastolic BP value BP(DIA) is determined at SA4, the control goes to SA5 to switch the pressure control valve 20 to the quick-deflation position and stop the air pump 26. Thus, Steps SA1 to SA5 correspond to the BP determining means 50.

Then, the control goes to SA6 corresponding to the pressing pressure determining means 52. At SA6, the control device 34 determines a blood-flow promoting pressure PP, based on the diastolic BP value BP(DIA) determined at SA4, according to the prescribed relationship shown in FIG. 3.

Next, the blood-flow promoting routine shown in FIG. 5 will be described. At SB1 of FIG. 5, one is added to a number counted by a timer t. Then, at SB2, a time represented by the number counted by the timer t has been increased up to the prescribed pressing period T₁ equal to one hour. While negative judgments are repeatedly made at SB2, Steps SB1 and SB2 are repeated, so that the number counted by the timer t is increased.

Meanwhile, if a positive judgment is made at SB2, the control goes to SB3 to reset the number counted by the timer T₁ to zero, and further to Steps SB4 to SB6 corresponding to the pressing means 54 so as to promote the blood flow of the patient.

At SB4, the air pump 26 is re-started and the pressure control valve 20 is switched to the pressure-supply position, so that the quick inflation of the cuff 16 is started. Then, at SB5, the control device 34 judges whether the cuff pressure PC has been increased up to the blood-flow promoting pressure PP determined at SA6 of FIG. 4. While negative judgments are repeatedly made at SB5, SB5 is repeated, so that the increasing of the cuff pressure PC is continued. Meanwhile, if a positive judgment is made at SB5, the control goes to SB6 to switch the pressure control valve 20 to the quick-deflation position and stop the air pump 26. The blood-flow promoting routine of FIG. 5 is periodically carried out till another START/STOP signal SS is supplied from the START/STOP switch 44 to the control device 34.

As is apparent from the foregoing description of the embodiment that employs the flow charts shown in FIGS. 4 and 5, the higher diastolic BP value BP(DIA) is actually determined at SA4, the higher blood-flow promoting pressure PP is determined at SA6 (i.e., the pressing pressure determining means 52). In other words, the lower diastolic BP value BP(DIA) is actually determined at SA4, the lower blood-flow promoting pressure PP is determined at SA6 (i.e., the pressing pressure determining means 52). Since, at Steps SB4 to SB6 (i.e., the pressing means 54), the ankle 14 is pressed with the cuff 16 whose pressure is equal to the blood-flow promoting pressure PP determined at SA6, the ankle 14 is pressed with an appropriate pressing pressure.

While the present invention has been described in its preferred embodiment, the present invention may be otherwise embodied.

For example, the cuff 16 of the venous thromboembolism preventing apparatus 10 shown in FIG. 1 is adapted to be worn on the ankle 14. However, it is possible to adapt the cuff 16 to be worn on a femoral portion, or a superior limb, such as an upper arm, of the patient. In addition, the venous thromboembolism preventing apparatus 10 shown in FIG. 1 employs the single cuff 16. However, it is possible to employ two or more cuffs. For example, two cuffs may be worn on a cremial portion of the patient such that the two cuffs are located on both sides of the calf of the cremial portion. In the case where two or more cuffs are employed, one of the cuffs that is located most downstream in the direction of flow of arterial blood is first used to press the body portion, and the most upstream cuff is last used to press the same, in order to flow venous blood toward the central side.

In addition, in the above-described venous thromboembolism preventing apparatus 10, the BP value BP itself is determined as a piece of BP-relating information. However, it is possible to obtain, in place of the BP value BP, different BP-relating information that relates to a BP value BP of the patient. For example, the BP-relating information may be pulse-wave-propagation-velocity-relating information relating to a velocity at which a pulse wave propagates between two prescribed portions of a living body, such as pulse-wave propagation velocity itself, or pulse-wave propagation time; an amplitude or an area of a pressure pulse wave that represents change of pressure in a blood vessel; or an amplitude or an area of a volumetric pulse wave that represents volume of blood. In the case where the BP-relating information other than the BP value BP itself is obtained, it is possible to directly determine the blood-flow promoting pressure PP based on the obtained BP-relating information. However, it is otherwise possible to additionally employ a relationship determining means for determining a relationship between estimated blood pressure BP and BP-relating information, based on the BP value BP determined by the BP determining means 50 and the obtained BP-relating information, and an estimated-BP determining means for determining, according to the thus determined relationship, an estimated BP value, based on each of successively obtained pieces of BP-relating information. In the last case, the pressing pressure determining means 52 uses the thus determined estimated BP value as the BP-relating information to determine a blood-flow promoting pressure PP.

In the case where the pulse-wave propagation time is obtained as the BP-relating information, Step SA6 of FIG. 4 employs a prescribed relationship between pressing pressure PP and pulse-wave propagation time, in which pressing pressure PP increases as pulse-wave propagation time decreases, because blood pressure BP increases as pulse-wave propagation time decreases.

In addition, in the above-described venous thromboembolism preventing apparatus 10, the cuff 16 is used to determine the BP value BP of the patient when the apparatus 10 is started, and the blood-flow promoting pressure PP is determined based on the BP value BP determined upon starting of the apparatus 10. Thus, while the venous thromboembolism preventing apparatus 10 is continuously operated, the blood-flow promoting pressure PP is not changed. However, it is possible to periodically obtain, at the pressing period T₁, a piece of BP-relating information such as a BP value BP or a piece of pulse-wave-propagation-velocity-relating information, and periodically determine a blood-flow promoting pressure PP at the pressing period T₁.

In addition, in the above-described venous thromboembolism preventing apparatus 10, the measurement of BP value BP, and the periodic blood-flow promoting pressing at the pressing period Ti are successively performed when the START/STOP switch 44 is operated in the state in which the apparatus 10 is at rest. However, it is possible to employ two separate switches for commanding a BP measurement and a blood-flow promoting pressing, respectively. In the latter case, the blood-flow promoting pressing may be performed independent of the BP measurement. To this end, the blood-flow promoting pressure PP is determined based on the BP value BP last determined by the BP determining means 50. In addition, the blood-flow promoting pressing may not be performed at the pressing period T₁. For example, a single blood-flow promoting pressing may be performed upon each operation of the corresponding switch.

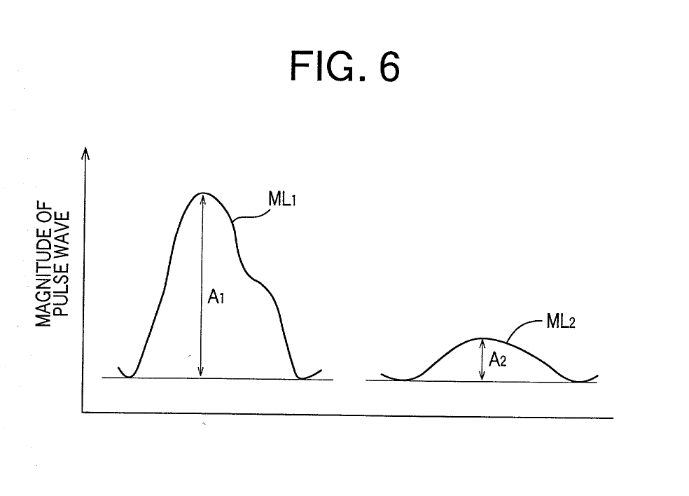

In addition, in the above-described venous thromboembolism preventing apparatus 10, the pressing means 54 periodically presses the ankle 14 at the pressing period T₁. However, the apparatus 10 may employ a phlebostasis judging means for judging whether an inferior limb of a patient has phlebostasis. In this case, only when the phlebostasis judging means judges that the inferior limb of the patient has phlebostasis, the pressing means 54 operates the cuff 16 to press the ankle 14. For example, the phlebostasis judging means may be one which finds phlebostasis when an absolute value of a rate of change of an amplitude A of each of heartbeat-synchronous pulses of an ankle pulse wave ML is greater than a reference value TH which is determined in advance based on experimental results. The ankle pulse wave ML is represented by the pulse-wave signal SM which is supplied by the pulse-wave filter circuit 30 in a state in which the pressing pressure of the cuff 16 is kept at from 20 to 30 mmHg. FIG. 6 shows respective ankle pulse waves ML which are detected when cnemial veins do not have phlebostasis and when cnemial veins have phlebostasis. More specifically described, a left-hand ankle pulse wave ML₁ is one which is detected when cnemial veins do not have phlebostasis, and a right-hand ankle pulse wave ML₂ is one which is detected when cnemial veins have phlebostasis. Since the amplitude A of each heartbeat-synchronous pulse of the ankle pulse wave ML decreases as the degree of phlebostasis of the creminal veins increases, it is possible to find phlebostasis based on the rate of change of amplitude A of the ankle pulse wave ML.

Hereinafter, there will be described other venous thromboembolism preventing apparatuses, as second to sixth embodiments of the present invention, each of which employs a phlebostasis judging means.

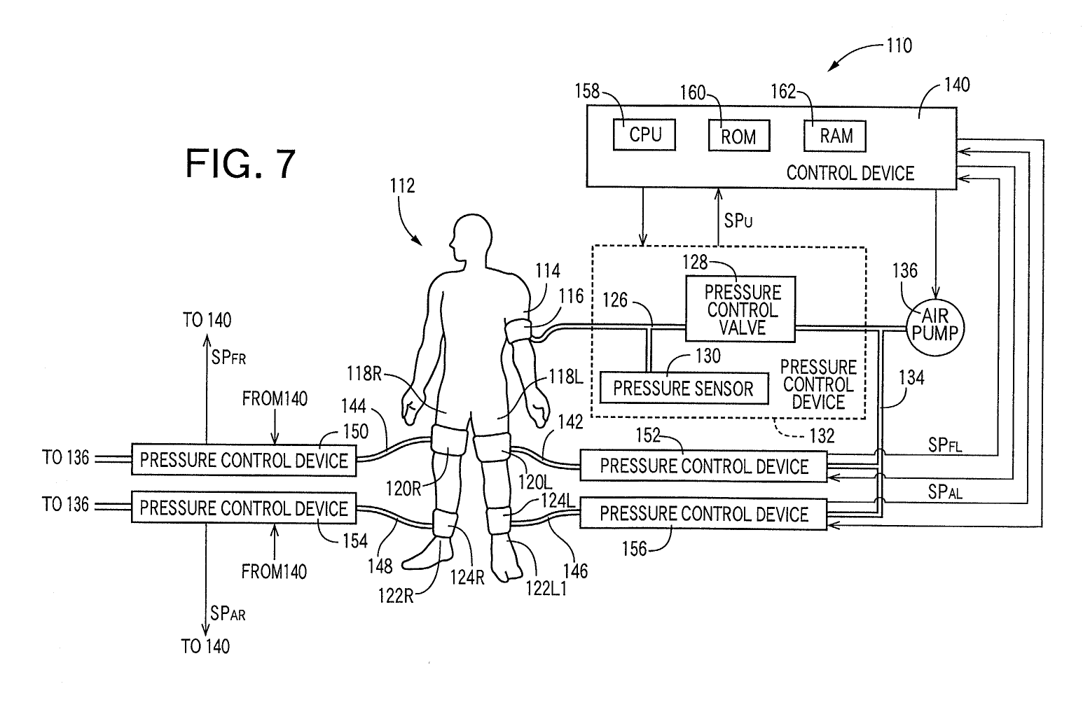

FIG. 7 shows a venous thromboembolism preventing apparatus 110 as the second embodiment of the present invention. In FIG. 1, the apparatus 110 includes an upper-arm cuff 116 which is adapted to be wound around an upper arm 114 of a patient 112 who is taking a face-up position; a right-femur cuff 120R and a left-femur cuff 120L which are adapted to be wound around a right femur 118R and a left femur 118L of the patient 112; and a right-ankle cuff 124R and a left-ankle cuff 124L which are adapted to be wound around a right ankle 122R and a left ankle 120L of the patient 112. Each of the cuffs 116, 120, 124 provides a pressing band which presses a body portion of the patient 12 around which the each cuff is wound, and the each cuff has the same structure as that of an inflatable cuff which is used in a blood-pressure measurement. More specifically described, each cuff 116, 120, 124 includes a belt-like outer bag which is formed of non-stretchable cloth or polyester and a rubber bag accommodated in the outer bag. The upper-arm cuff 116 has a width of 13 cm, each femur cuff 120 has a width of from 18 to 20 cm, and each ankle cuff 124 has a width of 12 cm.

The upper-arm cuff 116 is connected to a pressure control valve 128 and a pressure sensor 130 via a piping 126. The pressure control valve 128 and the pressure sensor 130 cooperate with each other to provide a pressure control device 132 which controls an air pressure in the upper-arm cuff 116. More specifically described, the pressure control valve 128 reduces, based on the pressure in the upper-arm cuff 116 detected by the pressure sensor 130, i.e., an upper-arm cuff pressure P_{U}, the pressure of a pressurized air supplied from the aim pump 136 via the piping 134, thereby controlling the pressure in the upper-arm cuff 116, i.e., the upper-arm cuff pressure P_{U}.

A pressure signal SP_{U} representing the upper-arm pressure PU detected by the pressure sensor 130, is supplied to an electronic control device 140 via an A/D converter, not shown.

The right and left femur cuffs 120 and the right and left ankle cuffs 124 are connected to respective pressure control devices 150, 152, 154, 156 via respective pipings 142, 144, 146, 148. Each of the pressure control devices 150, 152, 154, 156 has the same construction as that of the pressure control device 132, and is connected to the air pump 136 via the piping 134. Respective pressure signals SP_{FR}, SP_{FL}, SP_{AR}, SP_{AL} representing a right-femur cuff pressure P_{FR}, a left-femur cuff pressure P_{FL}, a right-ankle cuff pressure P_{AR}, and a left-ankle cuff pressure P_{AL}, detected by respective pressure sensors, not shown, of the pressure control devices 150, 152, 154, 156, are supplied to the control device 140 via respective A/D converters, not shown.

The control device 140 is provided by a so-called microcomputer including a CPU 158, a ROM 160, a RAM 162, and an I/O port, not shown. The CPU 158 processes signals according to control programs pre-stored in the ROM 160 by utilizing a temporary-storage function of the RAM 162, and supplies control signals to the air pump 136 and the pressure control devices 132, 150, 152, 154, 156 via the I/O port.

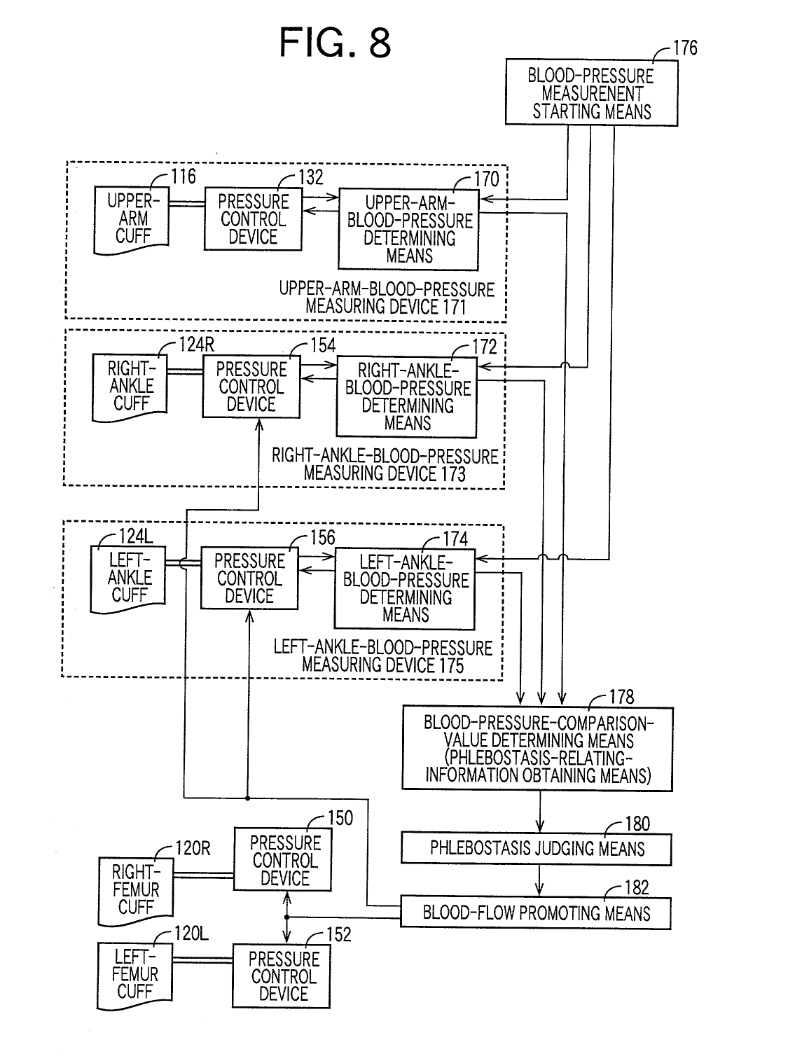

FIG. 8 is a block diagram for explaining essential functions of the control device 140. In the figure, an upper-arm blood-pressure (BP) determining means 170 includes a signal-filter means which subjects the upper-arm cuff-pressure signal SP_{U} supplied from the pressure control device 132, to a digital filter, and thereby provides a direct-current component of the cuff-pressure signal SP_{U}, i.e., a static-pressure signal DC_{U}, and an alternate-component of the signal SP_{U}, i.e., a pulse-wave signal AC_{U}. The upper-arm BP determining means 170 controls the air pump 136 and the pressure control device 132 at a prescribed BP-measurement starting period TB, SO that a pressing pressure P_{UDC} of the upper-arm cuff 116, represented by the static-pressure signal DC_{U}, is quickly increased up to a prescribed target pressure P_{M1} (e.g., about 180 mmHg) and subsequently the pressing pressure P_{UDC} is slowly decreased at a rate of 3 mmHg/sec. Based on the change of respective amplitudes of heartbeat-synchronous pulses of a pulse wave represented by the pulse-wave signal AC_{U} continuously obtained during the slow deflation of the upper-arm cuff 116, the BP determining means 170 determines a systolic BP value UBP_{SYS}, a mean BP value UBP_{MEAN}, and a diastolic BP value UBP_{DIA} of the upper arm 114 of the patient 112, according to a well-known oscillometric method. Since, in the present embodiment, the upper-arm cuff 116, the pressure control device 132, and the upper-arm BP determining means 170 cooperate with one another to measure the upper-arm BP values UBP, those elements 116, 132, 170 cooperate with one another to provide an upper-arm BP determining device 171.

A right-ankle BP determining means 172 has the same function as that of the upper-arm BP determining means 170. More specifically described, the right-ankle BP determining means 172 controls the air pump 136 and the pressure control device 154 at the BP-measurement starting period T_{B}, SO that a pressing pressure P_{ARDC} of the right-ankle cuff 124R, represented by the static-pressure signal DC_{AR} of the right-ankle cuff-pressure signal SP_{AR}, is quickly increased up to a prescribed target pressure P_{M2} (e.g., about 240 mmHg) and subsequently the pressing pressure P_{ARDC} is slowly decreased at a rate of 3 mmHg/sec. Based on the change of respective amplitudes of heartbeat-synchronous pulses of a pulse wave represented by the pulse-wave signal AC_{AR} continuously obtained during the slow deflation of the right-ankle cuff 124R, the BP determining means 172 determines a systolic BP value ARBP_{SYS}, a mean BP value ARBP_{MEAN}, and a diastolic BP value ARBP_{DIA} of the right ankle 122R of the patient 112, according to the well-known oscillometric method. Like the right-ankle BP determining means 172, a left-ankle BP determining means 174 determines a systolic BP value ALBP_{SYS}, a mean BP value ALBP_{MEAN}, and a diastolic BP value ALBP_{DIA} of the left ankle 122L of the patient 112. Thus, in the present embodiment, the right-ankle cuff 124R, the pressure control device 154, and the right-ankle BP determining means 172 cooperate with one another to provide a right-inferior-limb BP determining device 173; and the left-ankle cuff 124L, the pressure control device 156, and the left-ankle BP determining means 174 cooperate with one another to provide a left-inferior-limb BP determining device 175.

A BP-measurement starting means 176 periodically causes, at the prescribed BP-measurement starting period TB, the upper-arm BP determining means 170 to perform an upper-arm BP measuring operation, the right-ankle BP determining means 172 to perform a right-ankle BP measuring operation, and the left-ankle BP determining means 174 to perform a left-ankle BP measuring operation. The BP-measurement starting period TB is prescribed at, e.g., 30 minutes.

A BP-comparison-value determining means 178 determines a right-side comparison value Δ BP_{R} based on the upper-arm diastolic BP value UBP_{DIA} determined by the upper-arm BP determining means 170 and the diastolic BP value ARBP_{DIA} determined by the right-ankle BP determining means 172, and additionally determines a left-side comparison value Δ BP_{L} based on the upper-arm diastolic BP value UBP_{DIA} and the diastolic BP value ALBP_{DIA} determined by the lelf-ankle BP determining means 174. The right-side comparison value Δ BP_{R} may be a difference or a ratio of the upper-arm diastolic BP value UBP_{DIA} and the diastolic BP value ARBP_{DIA}, i.e., (ARBP_{DIA}-UBP_{DIA}) or (ARBP_{DIA}/UBP_{DIA}); and the left-side comparison value Δ BP_{L} may be a difference or a ratio of the upper-arm diastolic BP value UBP_{DIA} and the diastolic BP value ALBP_{DIA}, i.e., (ALBP_{DIA}-UBP_{DIA}) or (ALBP_{DIA}/UBP_{DIA}).

When cnemial veins, not shown, of the patient 112 have phlebostasis, the rate of decreasing of blood pressure after systolic blood pressure, at a portion of the patient 112 that is located on a proximal side of the cnemial veins, lowers because there is phlebostasis on a distal side of the cnemial veins. However, since the decreasing of blood pressure after systolic blood pressure lasts in a substantially constant duration only, the diastolic blood pressure at the proximal side of the cnemial veins increases. Thus, when the cnemial veins have phlebostasis, the diastolic blood pressure at the proximal side of the veins increases, but the influence of the phlebostasis decreases as the distance from the veins increases. Therefore, the upper arm 114 is free of the influence of phlebostasis of the cnemial veins. Accordingly, when phlebostasis occurs to the cnemial veins, the right-side or left-side BP comparison value Δ BP changes. Thus, the BP comparison value Δ BP is a sort of phlebostasis-relating information that changes in relation to phlebostasis of the cnemial veins of the patient 112; and the BP-comparison-value determining means 178 functions as a phlebostasis-relating-information obtaining means.

A phlebostasis judging means 180 judges that the cnemial veins of the patient 112 have phlebostasis, when the BP comparison value Δ BP determined by the BP-comparison-value determining means 178 is greater than a reference value TH_{BP} which is determined in advance based on experimental results. The reference value TH_{BP} can be said as an upper limit of a reference range which does not have a lower limit. As described previously, when phlebostasis occurs to the cnemial veins, the BP comparison value Δ BP changes. For example, in the case where the difference (ARBP_{DIA}-UBP_{DIA}) {or (ALBP_{DIA}-UBP_{DIA})} between the upper-arm diastolic BP value UBP_{DIA} and the right-ankle (or left-ankle) diastolic BP value ARBP_{DIA} (or ALBP_{DIA}) is determined as the BP comparison value Δ BP, the BP comparison value Δ BP increases when phlebostasis occurs to the cnemial veins. Hence, the phlebostasis judging means 180 judges that the cnemial veins have phlebostasis, when any one of the BP comparison values Δ BP iteratively determined by the BP-comparison-value determining means 178 is greater than the reference value TH_{BP}.

A blood-flow promoting means 182 operates, when the phlebostasis judging means 180 judges that the cnemial veins of the right leg of the patient 112 have phlebostasis, the pressure control device 154 to control the right-ankle cuff 124R so as to press the right ankle 122R and subsequently, while causing the cuff 124R to continue pressing the right ankle 122R, operates the pressure control device 150 to control the right-femur cuff 120R so as to press the right femur 118R. Then, the respective pressing pressures of the right-ankle cuff 124R and the right-femur cuff 120R are released. Thus, the flow of blood in the cnemial veins of the right leg is promoted.

Similarly, when the phlebostasis judging means 180 judges that the cnemial veins of the left leg of the patient 112 have phlebostasis, the blood-flow promoting means 182 operates the pressure control device 156 to control the left-ankle cuff 124L so as to press the left ankle 122L and subsequently, while causing the cuff 124L to continue pressing the left ankle 122L, operates the pressure control device 152 to control the left-femur cuff 120L so as to press the left femur 118L. Then, the respective pressing pressures of the left-ankle cuff 124L and the left-femur cuff 120L are released. Thus, the flow of blood in the cnemial veins of the left leg is promoted. However, the blood-flow promoting means 182 may be modified such that when the phlebostasis judging means 180 judges that the cnemial veins of at least one of the right and left legs of the patient 112 have phlebostasis, the blood-flow promoting means 182 operates the two pressure control devices 154, 156 to control the right-ankle and left-ankle cuffs 124R, 124L so as to press the right and left ankles 122R, 122L and subsequently, while causing the cuffs 124R, 124L to continue pressing the right and left ankles 122R, 122L, operates the two pressure control devices 150, 152 to control the right-femur and left-femur cuffs 120R, 120L so as to press the right and left femurs 118R, 118L. In this case, the flow of blood in the cnemial veins of each of the right and left legs is promoted.

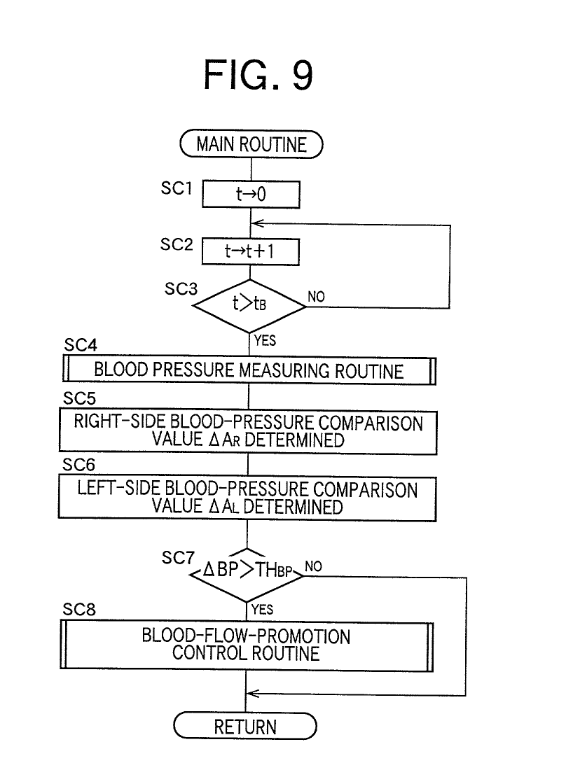

FIG. 9 shows a flow chart representing a control program according to which the control device 140 shown in FIG. 7 operates.

First, at Step SC1 (hereinafter, “Step” is omitted) of FIG. 9, a timer t is reset to zero and then, at SC2, one is added to the timer t.

At SC3, the control device 140 judges whether a time represented by a number counted by the timer t has exceeded the prescribed BP-measurement starting period T_{B}. If negative judgment is made at SC3, SC2 and SC3 are repeated, while an elapsing time is measured by the timer t.

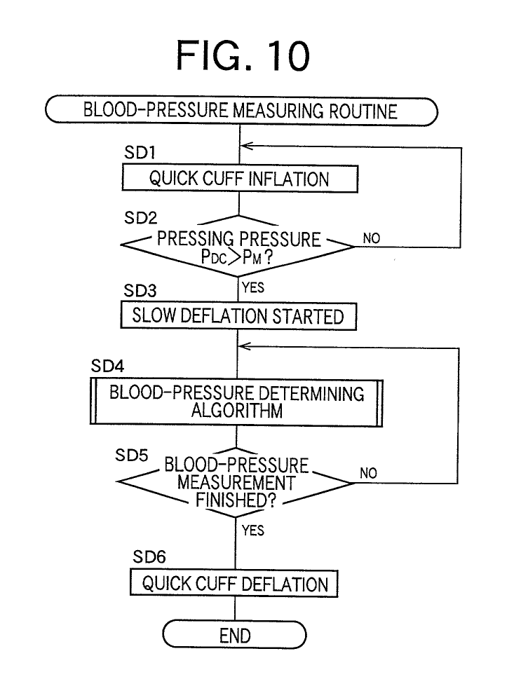

Meanwhile, if a positive judgment is made at SC3, the control goes to SC4, i.e., the BP measuring routine corresponding to the upper-arm BP determining means 170, the right-ankle BP determining means 172, and the left-ankle BP determining means 174. Thus, the BP value UBP at the upper arm 114, the BP value ARBP at the right ankle 122R, and the BP value ALBP at the left ankle 122L are simultaneously determined. The BP measuring routine at SC4 will be described below in detail by reference to the flow chart of FIG. 10.

At SD1 of the BP measuring routine of FIG. 10, the air pump 136 is started, and the pressure control devices 132, 154, 156 are operated, so that the respective pressing pressures P_{UDC}, P_{ARDC}, P_{ALDC} of the upper-arm cuff 116, the right-ankle cuff 124R, and the left-ankle cuff 124L are increased.

At SD2, the control device 140 judges whether each of the three pressing pressures P_{UDC}, P_{ADC}, P_{ALDC} has exceeded a corresponding one of the respective prescribed target pressures P_{M}. The target pressure P_{M1} corresponding to the upper arm 114 is prescribed at, e.g., 180 mmHg; and the target pressure P_{M2} corresponding to the ankles 22 is prescribed at, e.g., 240 mmHg.

While negative judgments are made at SD2, Steps SD1 and SD2 are repeated, while the pressing pressures P_{UDC}, P_{ARDC}, P_{ALDC} of the cuffs 116, 124 are continuously increased. Meanwhile, if a positive judgment is made at SD2, the control goes to SD3 to stop the air pump 136 and control the respective pressure control valves of the pressure control devices 132, 154, 156, so that the respective pressing pressures P_{UDC}, P_{ARDC}, P_{ALDC} of the upper-arm cuff 116, the right-ankle cuff 124R, and the left-ankle cuff 124L are slowly decreased at the rate of 3 mmHg/sec.

At SD4, the control device 140 subjects the upper-arm cuff-pressure signal SP_{U}, the right-ankle cuff-pressure signal SP_{AR}, and the left-ankle cuff-pressure signal SP_{AL} supplied thereto during the slow deflation of the cuffs 116, 124R, 124L, to the digital filter, so as to extract the respective pulse-wave signals AC_{U}, AC_{AR}, AC_{AL} from the three cuff-pressure signals SP_{U}, SP_{AR}, SP_{AL}. Based on a time-wise change of respective amplitudes of successive heartbeat-synchronous pulses of the pulse wave represented by each of the three pulse-wave signals AC_{U}, AC_{AR}, AC_{AL}, the control device 140 determines a systolic BP value BP_{SYS}, a mean BP value BP_{MEAN}, and a diastolic BP value BP_{DIA} of a corresponding one of the upper arm 114, the right ankle 122R, and the left ankle 122L of the patient 112, according to the well-known oscillometric BP determining algorithm.

At SD5, the control device 140 judges whether the current BP measurements have been finished, by judging whether the respective diastolic BP values BP_{DIA} of the upper arm 114, the right ankle 122R, and the left ankle 122L that are to be determined according to the oscillometric BP determining algorithm, last at SD4, have been determined. While negative judgments are made at SD5, Steps SD4 and SD5 are repeated, while the oscillometric BP determining algorithm is continuously carried out.

Meanwhile, if a positive judgment is made at SD5, the control goes to SD6 to control the respective pressure control valves of the pressure control devices 132, 154, 156 so as to quickly deflate the cuffs 116, 122R, 122L. Thus, the BP measuring routine is finished.

Back to FIG. 9, the control goes to SC5 and SC6 corresponding to the BP-comparison-value determining means 178. First, at SC5, a right-side BP comparison-value A BPR is determined by subtracting the upper-arm diastolic BP value UBP_{DIA} determined at SC4, from the right-ankle diastolic BP value ARBP_{DIA} and, then, at SC6, a left-side BP comparison-value Δ BP_{L} is determined by subtracting the upper-arm diastolic BP value UBP_{DIA} from the left-ankle diastolic BP value ALBP_{DIA}.

At SC7 corresponding to the phlebostasis judging means 180, the control device 140 judges whether either one of the two BP comparison-values Δ BP_{R}, Δ BP_{L} determined at SC5 and SC6 is greater than the prescribed reference value TH_{BP}. If neither of the two BP comparison-values Δ BP_{R}, Δ BP_{L} is greater than the prescribed reference value TH_{BP}, a negative judgment is made at SC7, and Steps SC1 to SC7 are repeated. Meanwhile, if either of the two BP comparison-values Δ BP_{R}, Δ BP_{L} is greater than the prescribed reference value TH_{BP}, a positive judgment is made at SC7, and the control goes to SC8 corresponding to the blood-flow-promotion control routine. Then, the control goes back to SC1.

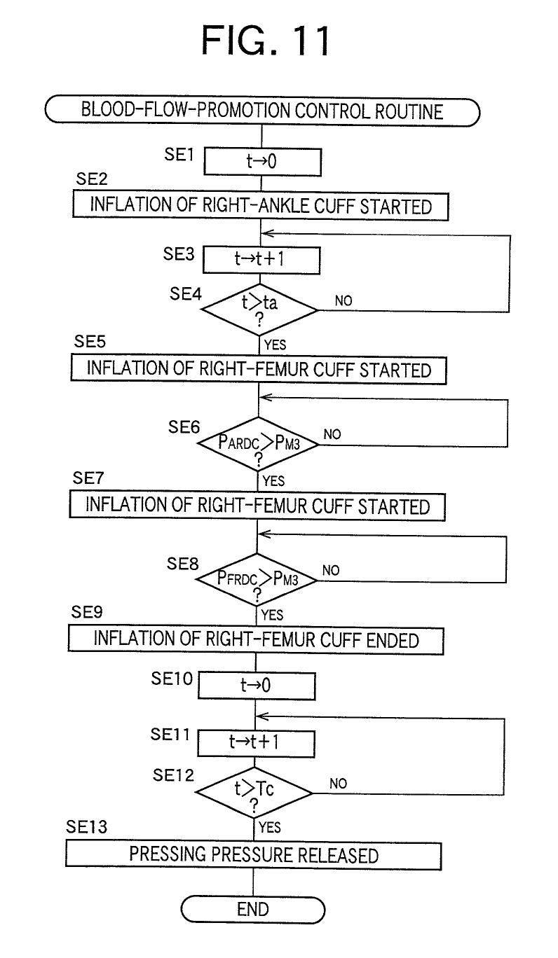

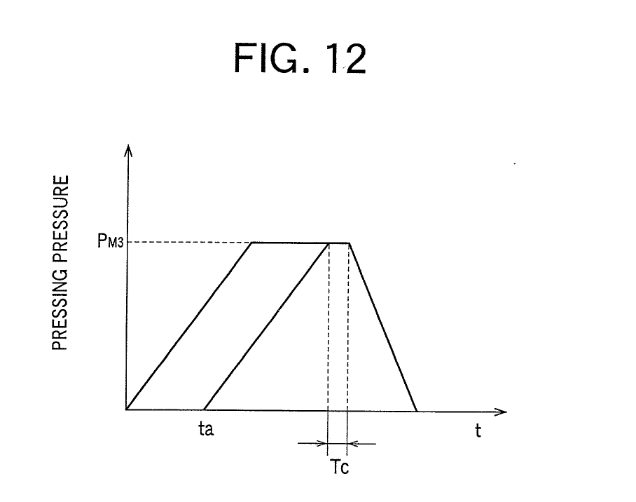

The blood-flow-promotion control routine at SC8 is carried out according to the flow chart of FIG. 11. FIG. 12 shows a graph representing respective time-wise changes of the respective pressing pressures P_{FRDC}, P_{ARDC} of the right-femur cuff 120R and the right-ankle cuff 124R according to the blood-flow-promotion control routine of FIG. 11. First, at SE1 of FIG. 11, a timer t is reset to zero and, at SE2, the control device 140 increases, for the right or left leg which has been judged to have phlebostasis at SC7 of FIG. 9 (here, it is assumed that the right leg has been judged to have phlebostasis), the pressing pressure P_{ARDC} of the right-ankle cuff 124R at a prescribed rate.

At SE3, one is added to the number counted by the timer t and, at SE4, the control device 140 judges whether a time represented by the number counted by the timer t has exceeded a prescribed elapsing time tₐ. While negative judgments are made at SE4, Steps SE3 and SE4 are repeated while an elapsing time is measured by the timer t. Meanwhile, if a positive judgment is made at SE4, the control goes to SE5 to increase, at a prescribed rate, the pressing pressure P_{FRDC} of the right-femur cuff 120R, i.e., the femur cuff 120 worn on the same leg as that on which the inflation of the ankle cuff 124 had been started at SE2.

At SE6, the control device 140 judges whether the pressing pressure P_{ARDC} of the right-ankle cuff 124R has exceeded a prescribed target pressure P_{M3}. If a negative judgment is made at SE6, SE6 is repeated while the increasing of the pressing pressure P_{ARDC} of the right-ankle cuff 124R is continued. Meanwhile, if a positive judgment is made at SE6, the control goes to SE7 to operate the pressure control device 154 to stop the inflation of the right-ankle cuff 124R and keep the pressing pressure P_{ARDC} at the prescribed target pressure P_{M3}. The target pressure P_{M3} is prescribed at a pressure, e.g., 60 mmHg, that is higher than a normal blood pressure in cnemial veins.

Next, at SE8, the control device 140 judges whether the pressing pressure P_{FRDC} of the right-femur cuff 120R has exceeded the prescribed target pressure P_{M3}. If a negative judgment is made at SE8, SE8 is repeated while the increasing of the pressing pressure P_{FRDC} of the right-femur cuff 120R is continued. Meanwhile, if a positive judgment is made at SE8, the control goes to SE9 to operate the pressure control device 150 to stop the inflation of the right-femur cuff 120R and keep the pressing pressure P_{FRDC} at the prescribed target pressure P_{M3}.

Then, at SE10, the timer t is reset again to zero and, at SE11, one is added to the number counted by the timer t. At SE12, the control device 140 judges whether the time represented by the number counted by the timer t has exceeded a prescribed pressure-keep duration T_{C}. While negative judgments are made at SE12, Steps SE11 and SE12 are repeated while an elapsing time is measured by the timer t. Meanwhile, if a positive judgment is made at SE12, the control goes to SE13 to release the respective pressing pressures P_{ARDC}, P_{FRDC} of the right-ankle cuff 124R and the right-femur cuff 120R. Thus, the blood-flow-promotion control routine is finished.

As is apparent from the foregoing description of the second embodiment, the phlebostasis judging means 180 (SC7) judges that the cnemial veins have phlebostasis, based on whether the BP comparison value Δ BP determined by the BP-comparison-value determining means 178 (SAC and SAC) is greater than the prescribed reference value TH_{BP}. Only when the cnemial veins is judged to have phlebostasis, the blood-flow promoting means 182 (SC8) operates the ankle cuff 124 and the femur cuff 120, in this order, to sequentially press the inferior limb and thereby promote the flow of blood in the cnemial veins. Thus, the number of pressing times can be minimized.

Next, there will be described a third embodiment of the present invention. The same reference numerals as used in the second embodiment are used to designate the corresponding elements and parts of the third embodiment.

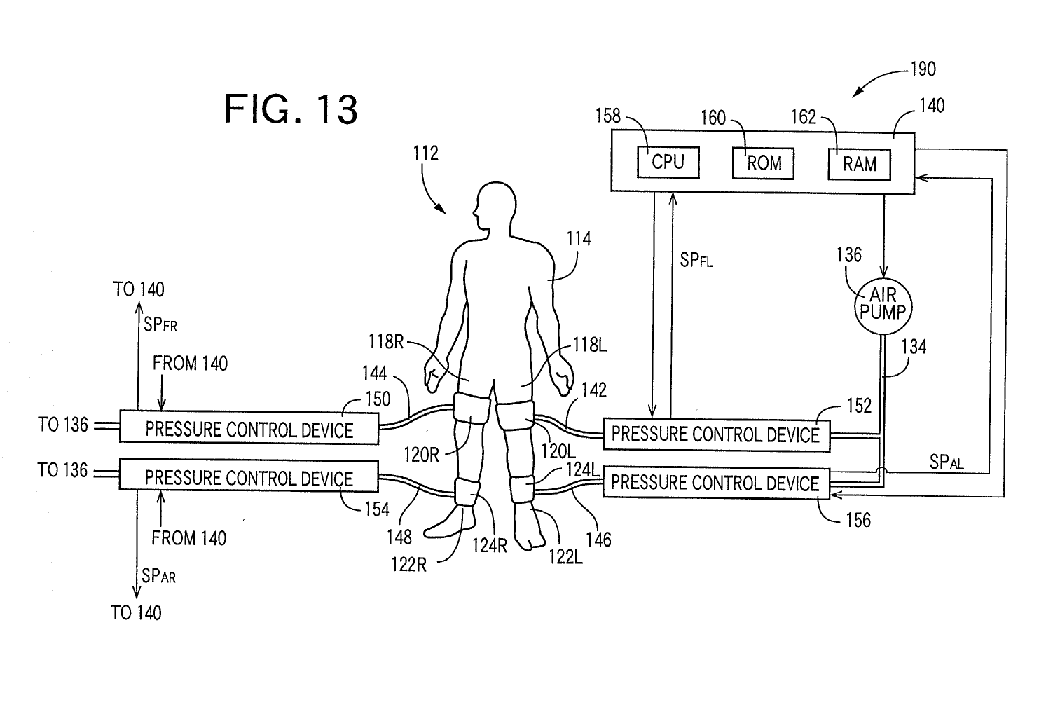

FIG. 13 shows another venous thromboembolism preventing apparatus 190 as the third embodiment of the present invention. The present apparatus 190 is basically similar to the preceding apparatus 110 and is different from the same 110 only in that the present apparatus 190 does not employ the upper-arm cuff 116, or the pressure control device 132 connected to the cuff 116, and that a control device 140 of the present apparatus 190 have different functions than those of the control device 140 of the preceding apparatus 110.

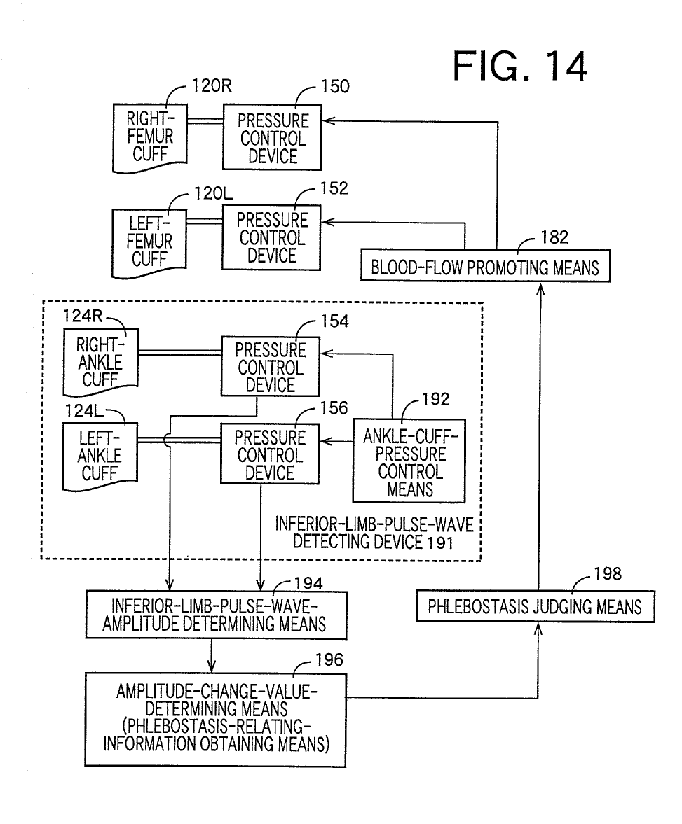

FIG. 14 is a block diagram for explaining essential functions of the control device 140 of the present apparatus 190. In the figure, an ankle-cuff-pressure control means 192 controls the air pump 136 and the pressure control devices 154, 156, so that respective pressing pressures P_{ARDC}, P_{ALDC} of the right-ankle and left-ankle cuff 124R, 124L are increased up to a prescribed target pressure P_{M4} (e.g., from 20 to 30 mmHg) that is sufficiently lower than a mean BP value BP_{MEAN} of the patient 112. After the pressing pressures P_{ARDC}, P_{ALDC} are kept at the prescribed target pressure P_{M4} for a prescribed time duration, those pressures P_{ARDC}, P_{ALDC} are decreased to the atmospheric pressure. In the state in which the respective pressing pressures P_{ARDC}, P_{ALDC} of the right-ankle and left-ankle cuff 124R, 124L are kept at the prescribed target pressure P_{M4}, the respective pressure sensors (not shown) of the pressure control devices 154, 156 detect respective ankle pulse waves ML_{R}, ML_{L} as respective pressure pulse waves which are produced by respective arteries of the right and left ankles and are propagated to respective ankle skins or the right-ankle and left-ankle cuffs 124R, 124L. Thus, in the present embodiment, the right-ankle cuff 124R, the pressure control device 154, and the ankle-cuff-pressure control means 192 cooperate with one another to provide a first pressure-pulse-wave detecting device or a right-inferior-limb-pulse-wave detecting device; and the left-ankle cuff 124L, the pressure control device 156, and the ankle-cuff-pressure control means 192 cooperate with one another to provide a second pressure-pulse-wave detecting device or a left-inferior-limb-pulse-wave detecting device.

An inferior-limb-pulse-wave-amplitude determining means 194 iteratively determines an amplitude A_{R} of each of successive heartbeat-synchronous pulses of the right-ankle pulse wave ML_{R}, and an amplitude A_{L} of each of successive heartbeat-synchronous pulses of the left-ankle pulse wave ML_{L}, which pulse waves are detected by the respective not-shown pressure sensors of the pressure control devices 154, 156 in the state in which the respective pressing pressures P_{ARDC}, P_{ALDC} of the right-ankle and left-ankle cuff 124R, 124L are kept at the prescribed target pressure P_{M4} by the ankle-cuff-pressure control means 192.

FIG. 6 shows respective ankle pulse waves ML which are detected by the above-described right or left inferior-limb-pulse-wave detecting device when cnemial veins do not have phlebostasis and when cnemial veins have phlebostasis. More specifically described, a left-hand ankle pulse wave ML₁ is one which is detected when cnemial veins do not have phlebostasis, and a right-hand ankle pulse wave ML₂ is one which is detected when cnemial veins have phlebostasis. As shown in FIG. 6, the amplitude A of each heartbeat-synchronous pulse of the ankle pulse wave ML, i.e., the pressure pulse wave propagated to the ankle cuff 124 wound around the ankle 122 of the patient 112 decreases as the degree of phlebostasis of the creminal veins of the patient 112 increases.

The ankle pulse wave ML propagated to the ankle cuff 124 consists essentially of the pressure pulse wave produced by the arteries of the ankle 122. Those arteries are located upstream of the cnemial veins. When phlebostasis occurs to the cnemial veins, the pulsation of the arteries located upstream of those veins decreases. Therefore, when the cnemial veins have phlebostasis, the amplitude A of the ankle pulse wave ML decreases. However, the influence of phlebostasis decreases as the distance from the veins increases. Thus, the inferior-limb-pulse-wave detecting device needs to detect a pulse wave from a distal-side portion of an inferior limb that is located on a distal side of its knee, more preferably, its calf as in the third embodiment.

An amplitude-change-value determining means 196 determines a change value Δ A_{R} of each of the amplitudes A_{R} of the right-ankle pulse wave ML_{R} that are iteratively determined by the inferior-limb-pulse-wave-amplitude determining means 194, and a change value Δ A_{L} of each of the amplitudes A_{L} of the left-ankle pulse wave ML_{L} that are iteratively determined by the determining means 194. The amplitude change value Δ A_{R}, Δ A_{L} may be a difference, or a ratio, of each current amplitude A from or to at least one past amplitude determined at least one prescribed time before, or a difference, or a ratio, of each current amplitude A from or to at least one initial amplitude determined immediately after the current operation of the apparatus 190 is initiated. When the degree of phlebostasis of the cnemial veins of the right leg increases, the amplitude change value Δ A_{R} changes. Similarly, when the degree of phlebostasis of the cnemial veins of the left leg increases, the amplitude change value Δ A_{L} changes. Thus, the amplitude change value Δ A (Δ A_{R} or Δ A_{L}) is a sort of phlebostasis-relating information, and the amplitude-change-value determining means 196 functions as the phlebostasis-relating-information obtaining means.

A phlebostasis judging means 198 judges that the cnemial veins of the patient 112 have phlebostasis, when the amplitude change value Δ A determined by the amplitude-change-value determining means 196 is smaller than a reference value TH_{A} which is determined in advance based on experimental results. The reference value TH_{A} can be said as a lower limit of a reference range which does not have an upper limit. When the phlebostasis judging means 198 judges that the cnemial veins of the right or left leg of the patient 112 have phlebostasis, a blood-flow promoting means 182 operates, as described previously, to promote the flow of blood in the cnemial veins of the leg in problem only, or both the right and left legs.

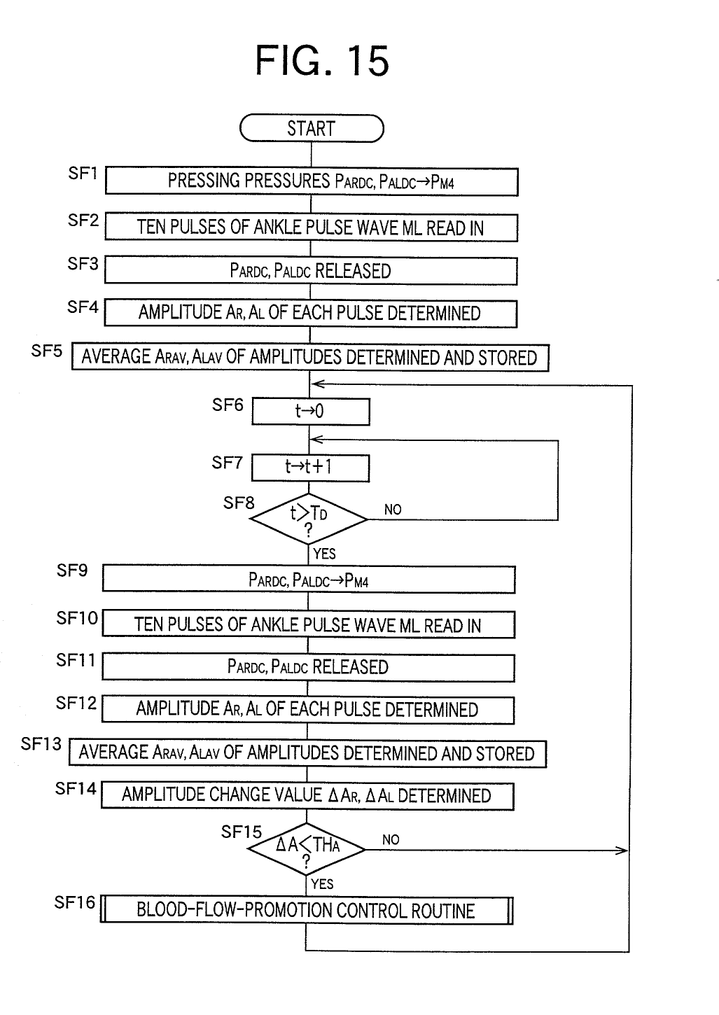

FIG. 15 shows a flow chart representing a control program according to which the control device 140 shown in FIG. 13 operates.

First, at Step SF1 corresponding to the ankle-cuff-pressure control means 192, the control device 140 operates the pressure control devices 154, 156 to increase the respective pressing pressures P_{ARDC}, P_{ALDC} of the right and left ankle cuffs 124R, 124L up to the target pressure P_{M4}.

At SF2, in the state established at SF1, the control device 140 read in ten heartbeat-synchronous pulses of the right-ankle pulse wave ML_{R}, and ten heartbeat-synchronous pulses of the left-ankle pulse wave ML_{L}, which pulse waves are detected by the respective not-shown pressure sensors of the pressure control devices 154, 156. However, it is possible to read in a single heartbeat-synchronous pulse, or a prescribed number (greater than one) of pulses, or read in pulses in a prescribed time duration.

After the ten pulses of the right-ankle pulse wave ML_{R} and the ten pulses of the left-ankle pulse wave ML_{F} are read in at SF2, the control goes to SF3 to operate the pressure control devices 154, 156 to decrease the respective pressing pressures of the two ankle cuffs 124R, 124L down to the atmospheric pressure. Then, at SF4 corresponding to the inferior-limb-pulse-wave-amplitude determining means 194, the control device 140 determines respective amplitudes A_{R} of the ten pulses of the right-ankle pulse wave ML_{R}, and respective amplitudes A_{L} of the ten pulses of the left-ankle pulse wave ML_{F}, which pulses had been read in at SF2. Subsequently, at SF5, the control device 140 determines an average A_{RAV} of the ten amplitudes A_{R}, and an average A_{LAV} of the ten amplitudes A_{L}, which amplitudes had been determined at SF4. The thus determined average A_{RAV}, A_{LAV} are stored in a prescribed portion of the RAM 162.

At SF6, a timer t is reset to zero and, at SF7, one is added to a number counted by the timer t. Subsequently, at SF8, the control device 140 judges whether a time represented by the number counted by the timer t has exceeded a prescribed judging period T_{D}. The judging period T_{D} is employed to periodically judge whether cnemial veins have phlebostasis. While negative judgments are made at SF8, Steps SF7 and SF8 are repeated, while an elapsing time is measured by the timer t.

Meanwhile, if a positive judgment is made at SF8, the control goes to Steps SF9 to SF13 that are the same as Steps SF1 to SF5. In short, at SF9, the respective pressing pressures P_{ARDC}, P_{ALDC} of the right and left ankle cuffs 124R, 124L are increased up to the target pressure P_{M4}. At SF10, ten pulses of the right-ankle pulse wave ML_{R} and ten pulses of the left-ankle pulse wave ML_{L} are read in. At SF11, the respective pressing pressures of the two ankle cuffs 124R, 124L are decreased down to the atmospheric pressure. At SF12, respective amplitudes A_{R} of the ten pulses of the right-ankle pulse wave ML_{R} and respective amplitudes A_{L} of the ten pulses of the left-ankle pulse wave ML_{F} are determined. And, at SF13, an average A_{RAV} of the ten amplitudes A_{R} and an average A_{LAV} of the ten amplitudes A_{L} are determined, and are stored in the RAM 162.

At SF14 corresponding to the amplitude-change-value determining means 196, the control device 140 determines, as the right-inferior-limb amplitude change value Δ A_{R}, a ratio of the current average A_{RAV} determined and stored at SF13 to the initial average A_{RAV} determined and stored at SF5, and determines, as the left-inferior-limb amplitude change value Δ A_{L}, a ratio of the current average A_{LAV} determined and stored at SF13 to the initial average A_{LAV} determined and stored at SF5.

Next, at SF15 corresponding to the phlebostasis judging means 198, the control device 140 judges whether at least one of the two amplitude change value Δ A_{R}, Δ A_{L} determined at SF14 is smaller than the prescribed reference value TH_{A}. As the degree of phlebostasis of the cnemial veins increases, the amplitude A determined at SF12 decreases and accordingly the amplitude change value Δ A (Δ A_{R} or Δ A_{L}) determined at SF14 decreases. Therefore, the phlebostasis can be found by judging whether the amplitude change value Δ A_{R} or Δ A_{L} is smaller than the prescribed reference value TH_{A}.

If a negative judgment is made at SF15, that is, if the cnemial veins do not have phlebostasis, Step SF6 and the following steps are repeated, so that at the judging period T_{D}, it is periodically judged whether the cnemial veins have phlebostasis. Meanwhile, if a positive judgment is made at SF15, i.e., if the cnemial veins have phlebostasis, then the control goes to SF16, i.e., the previously-described blood-flow-promotion control routine shown in FIG. 11, so that the flow of blood in the cnemial veins is promoted. Then, the control goes back to Step SF6 and the following steps.

It emerges from the foregoing description of the third embodiment that the phlebostasis judging means 198 (SF15) judges that the cnemial veins of the patient 112 have phlebostasis, based on whether the amplitude change value Δ A determined by the amplitude-change-value determining means 196 (SF14) is smaller than the prescribed reference value TH_{A}. Only when the cnemial veins of the patient 112 is judged to have phlebostasis, the blood-flow promoting means 182 (SF16) operates the ankle cuff 124 and the femur cuff 120, in this order, to sequentially press the inferior limb and thereby promote the flow of blood in the cnemial veins of the inferior limb. Thus, the number of pressing times can be minimized.

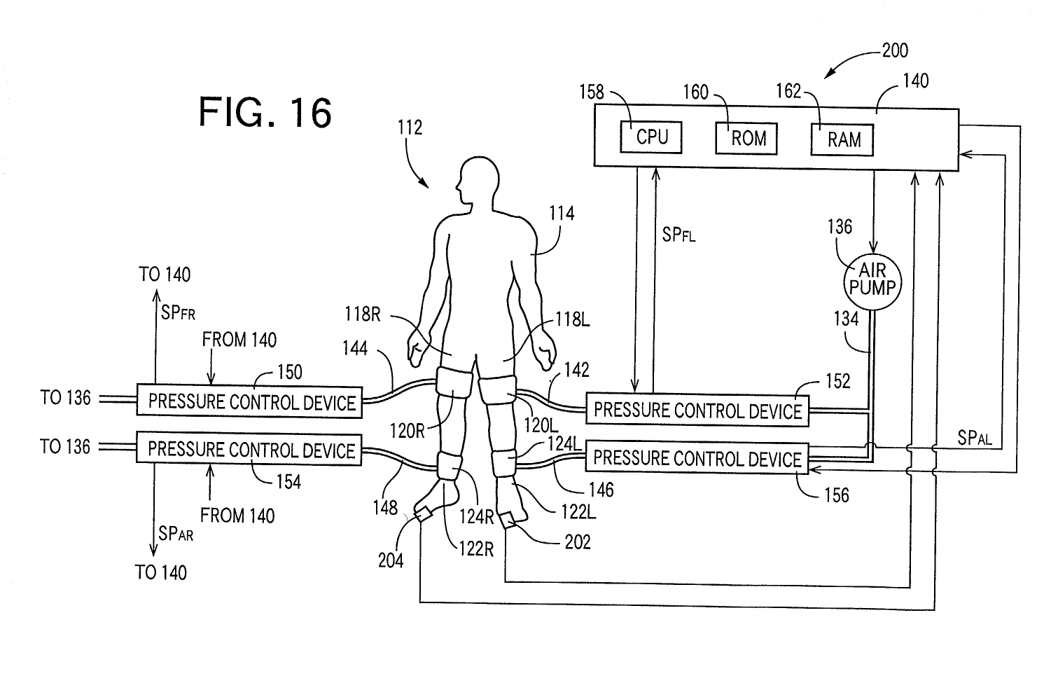

Next, there will be described a fourth embodiment of the present invention. FIG. 16 shows another venous thromboembolism preventing apparatus 200, as the fourth embodiment of the invention, that is different from the preceding apparatuses 110, 190 in that the present apparatus 200 employs two photoelectric-pulse-wave sensors 202, 204.

In the present embodiment, each of the two photoelectric-pulse-wave sensors 202, 204 provides an inferior-limb-pulse-wave detecting device. The two sensors 202, 204 are adapted to be worn on respective big toes of left and right feet of the patient 112. Each sensor 202, 204 includes a housing, not shown, which can accommodates a big toe of a foot; and a light emitter and a light receiver, both not shown. The light emitter is a light source which emits, toward the skin surface of the patient 112, a red or infrared light having a wavelength that can be reflected by hemoglobin, more preferably, a light having a wavelength of about 800 nm that is not influenced by blood oxygen saturation. The light receiver detects a light scattered by the skin tissue and generates a photoelectric-pulse-wave signal representing a photoelectric pulse wave, i.e., a volumetric pulse wave corresponding to a volume of blood present in capillaries of the skin tissue. The respective photoelectric-pulse-wave signals generated by the photoelectric-pulse-wave sensors 202, 204 are supplied to a control device 140 via respective A/D converters, not shown.

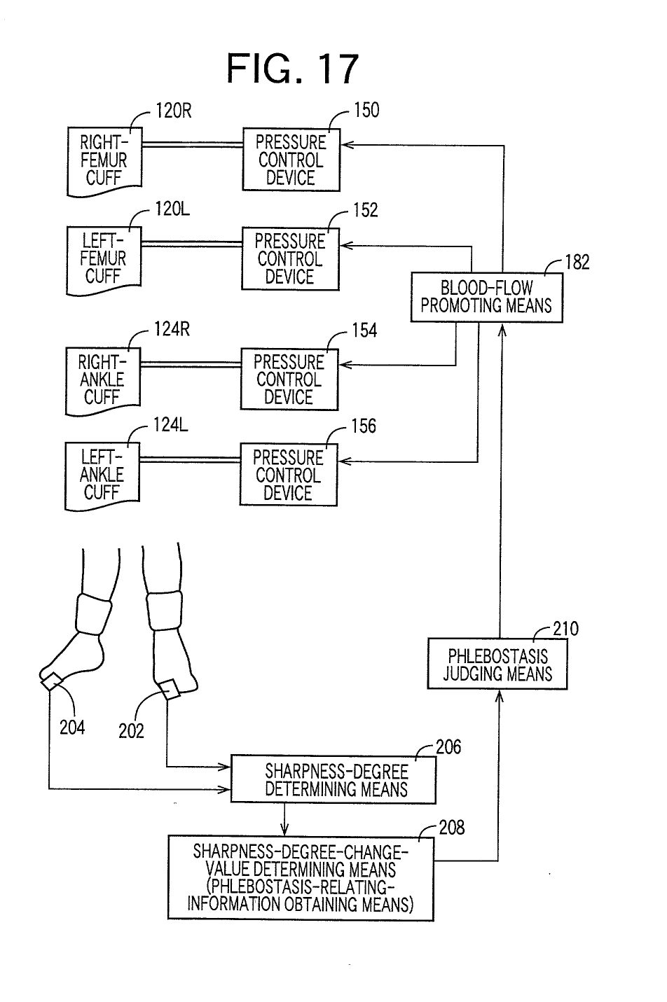

FIG. 17 is a block diagram for explaining essential functions of the control device 140 of the present apparatus 200. The functions of the control device 140 of the fourth embodiment differ from those of the control devices 140 of the second or third embodiment, in that the present control device 140 includes a sharpness-degree determining means 206 which iteratively determines a degree of sharpness of each of successive heartbeat-synchronous pulses of the photoelectric-pulse-wave signal generated by each of the photoelectric-pulse-wave sensors 202, 204 each as the inferior-limb-pulse-wave detecting device; and a sharpness-degree-change-value determining means 208 which iteratively determines a change value of each of the sharpness degrees iteratively determined by the sharpness-degree determining means 206.

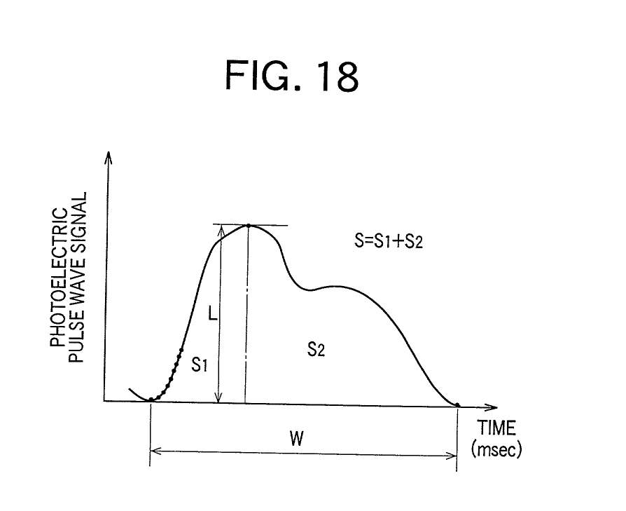

The degree of sharpness means a degree of upward projection of each heartbeat-synchronous pulse of the photoelectric pulse wave. For example, as shown in FIG. 18, an area S (=S₁+S₂) of each heartbeat-synchronous pulse of the photoelectric pulse wave can be determined by integrating the photoelectric-pulse-wave signal with respect to a pulse period W. The degree of sharpness may be expressed by using a ratio, S/(W×L), of the area S to the product of the pulse period W and a peak height L, i.e., a normalized pulse area VR; a normalized value of a first half area S₁ or a normalized value of a second half area S₂ (the first and second half areas S₁, S₂ are divided from each other at the peak indicated at one-dot chain line); or a normalized value, I/W, of a width I of each pulse at a height equal to L×(2/3). The normalized pulse area VR that may be called as % MAP may be expressed by using a ratio of a height G of a gravity center of the pulse area S to the peak height L, i.e., pulse pressure.

The sharpness-degree-change-value determining means 208 iteratively determines a change value of each of the sharpness degrees iteratively determined by the sharpness-degree determining means 206. The change value may be a difference, or a ratio, of each current sharpness degree from or to at least one past sharpness degree determined at least one prescribed time before, or a difference, or a ratio, of each current sharpness degree from or to at least one initial sharpness degree determined immediately after the current operation of the apparatus 200 is initiated. As the degree of phlebostasis of the cnemial veins of each leg increases, the flow of blood in the veins present on the distal side of its knee decreases. Accordingly, the shape of each heartbeat-synchronous pulse of the photoelectric pulse wave becomes duller. Since the photoelectric-pulse-wave sensors 202, 204 are worn on the respective big toes of the two feet located on the distal side of the respective knees of the two legs, the sharpness degree of the photoelectric pulse wave detected by each of the two sensors 202, 204 increases as the degree of phlebostasis of the cnemial veins of the corresponding leg increases. Thus, the sharpness-degree change value is a sort of phlebostasis-relating information which changes in relation with the degree of phlebostasis of the cnemial veins; and the sharpness-degree-change-value determining means 208 functions as the phlebostasis-relating-information obtaining means.

A phlebostasis judging means 210 judges that the cnemial veins of the patient 112 have phlebostasis, when the sharpness-degree change value determined by the sharpness-degree-change-value determining means 208 is greater than a reference value which is determined in advance based on experimental results. This reference value can be said as an upper limit of a reference range which does not have a lower limit. As the degree of phlebostasis of the cnemial veins increases, the sharpness-degree change value determined by the sharpness-degree-change-value determining means 208 increases. Therefore, the sharpness-degree change value can be used to find the phlebostasis of the cnemial veins.

When the phlebostasis judging means 210 judges that the cnemial veins of the right or left leg of the patient 112 have phlebostasis, a blood-flow promoting means 182 operates, as described previously, to promote the flow of blood in the cnemial veins of the leg in problem only, or both the right and left legs.

It emerges from the foregoing description of the fourth embodiment that the phlebostasis judging means 210 judges that the cnemial veins of the patient 112 have phlebostasis, based on whether the sharpness-degree change value determined by the sharpness-degree-change-value determining means 208 is greater than the prescribed reference value. Only when the cnemial veins of the patient 112 is judged to have phlebostasis, the blood-flow promoting means 182 operates the ankle cuff 124 and the femur cuff 120, in this order, to sequentially press the inferior limb and thereby promote the flow of blood in the cnemial veins of the inferior limb. Thus, the number of pressing times can be minimized.

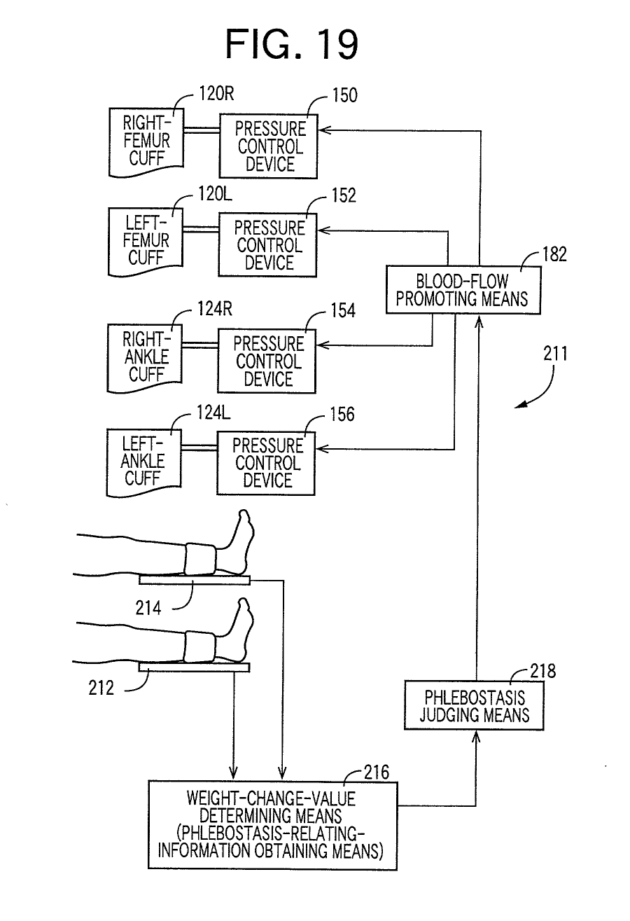

Next, there will be described a fifth embodiment of the present invention. FIG. 19 shows essential functions of a control device 140 of another venous thromboembolism preventing apparatus 211, as the fifth embodiment of the invention, that is different from the preceding apparatuses 110, 190, 200 in that the present apparatus 211 employs, in place of the two photoelectric-pulse-wave sensors 202, 204, two weight measuring devices 212, 214.

The two weight measuring devices 212, 214 support respective under-knee portions of a right leg and a left leg of a patient 112. Here, an under-knee portion may be the entirety of a portion of a leg that is located on a distal side of its knee, or a part of that entire portion. Each of the weight measuring devices 212, 214 is provided by, e.g., a load cell. Each device 212, 214 iteratively measures a weight of an under-knee portion of a leg of the patient 112 that is placed thereon, and supplies a weight signal representing the detected weight, to the control device 140.

A weight-change-value determining means 216 iteratively determines a change value of each of the weights iteratively measured by each of the two weight measuring devices 212, 214. The change value may be an amount of change, or a rate of change, of each current weight from at least one prior weight. When phlebostasis occurs to the veins of an inferior limb, the weight of the under-knee portion of the inferior limb increases by the amount of blood staying in the veins, and accordingly the change value of each current weight increases. Therefore, the change value of the weight of the under-knee portion is a sort of phlebostasis-relating information, and the weight-change-value determining means 216 functions as the phlebostasis-relating-information obtaining means.

A phlebostasis judging means 218 judges that the cnemial veins of the patient 112 have phlebostasis, when the weight change value determined by the weight-change-value determining means 216 is greater than a reference value which is determined in advance based on experimental results. This reference value can be said as an upper limit of a reference range which does not have a lower limit. As the degree of phlebostasis of the cnemial veins increases, the weight change value determined by the weight-change-value determining means 216 increases. Therefore, the weight change value can be used to find the phlebostasis of the cnemial veins.

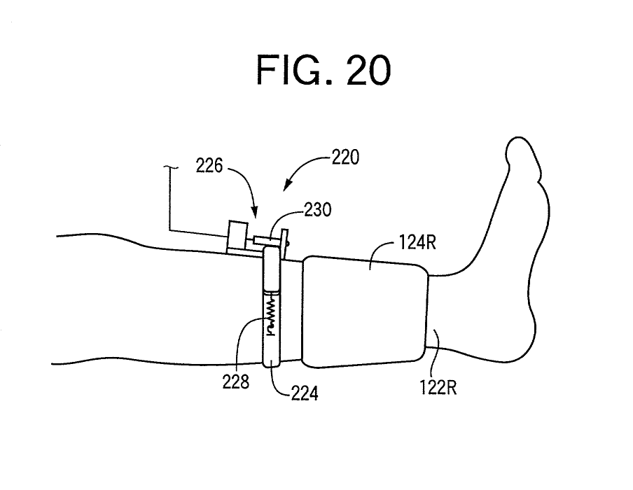

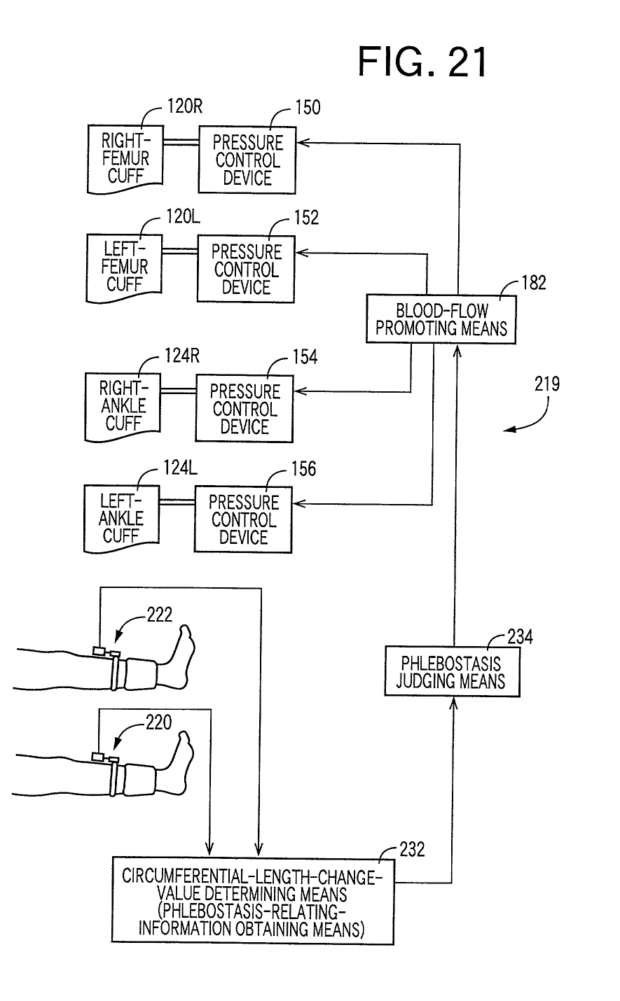

Next, there will be described a sixth embodiment of the present invention. FIG. 20 shows one of two circumferential-length detecting devices 220, 222 of another venous thromboembolism preventing apparatus 219, as the sixth embodiment of the invention, that is different from the preceding apparatuses 110, 190, 200, 211. The one detecting device 220, shown in FIG. 20, is adapted to be worn on a right leg of a patient 112, and the other detecting device 222, shown in FIG. 21, is adapted to be worn on a left leg of the patient 112. The two detecting devices 220, 222 have an identical construction. The present apparatus 219 differs from the fifth apparatus 211 in that the former apparatus 219 employs, in place of the weight measuring devices 212, 214, the two circumferential-length detecting devices 220, 222.

The right circumferential-length detecting device 220, as a representative of the two devices 220, 222, includes a band 224 which has a considerably small width and is adapted to be wound around a portion of the right leg of the patient 112 that is located between its knee and its ankle 122; and a rotary-type position sensor 226. A spring 228 which has a considerably small spring constant is fixed, at one end thereof, to an outer-side end portion of the band 224, and the other end of the spring 228 is detachably attached to a prescribed position on an outer surface of the band 224. The spring constant of the spring 228 is prescribed at a minimal value that can keep the band 224 wound around the right leg. Therefore, the leg receives substantially no pressure from the band 224. The rotary-type position sensor 226 is provided at an inner-side end portion of the band 224, and includes a rotation member 230 whose lengthwise direction is parallel to the widthwise direction of the band 224 and which is adapted to contact the outer surface of the band 224. Since the rotation member 230 is continuously rotated as the band 224 is continuously moved in a circumferential direction of the right leg, the rotary-type position sensor 226 continuously detects an total amount of movement of the band 224 in the circumferential direction of the right leg, i.e., a total amount of change of a circumferential length of the portion of the right leg around which the band 224 is wound. The position sensor 226 continuously supplies a signal representing the total movement amount of the band 224 in the circumferential direction of the right leg, to a control device 140.

FIG. 21 shows essential functions of the control device 140 of the present venous thromboembolism preventing apparatus 219. A circumferential-length-change determining means 232 iteratively determines, based on the signal continuously supplied from each of the right and left circumferential-length detecting devices 220, 222, a change value of the circumferential length of a corresponding one of the right and left legs. The change value may be an amount of change, or a rate of change, of each current circumferential length from at least one prior circumferential length. When phlebostasis occurs to the veins of an inferior limb, the circumferential length of any portion of the inferior limb located between its knee and its ankle 122 increases because of the amount of blood staying in the veins. Therefore, the change value of the circumferential length of the inferior limb is a sort of phlebostasis-relating information, and the circumferential-length-change-value determining means 232 functions as the phlebostasis-relating-information obtaining means.

A phlebostasis judging means 234 judges that the veins of the inferior limb of the patient 112 have phlebostasis, when the circumferential-length change value determined by the circumferential-length-change-value determining means 232 is greater than a reference value which is determined in advance based on experimental results. This reference value can be said as an upper limit of a reference range which does not have a lower limit. As the degree of phlebostasis of the cnemial veins increases, the circumferential-length change value determined by the circumferential-length-change-value determining means 232 increases. Therefore, the circumferential-length change value can be used to find the phlebostasis of the cnemial veins.

While the present invention has been described in its preferred embodiments by reference to the drawings, it is to be understood that the invention may otherwise be embodied.

For example, in the second embodiment shown in FIG. 7, the inferior-limb BP measuring device 173, 175 is of a type which measures a BP value of the ankle 122 of the patient 112. However, the inferior-limb BP measuring device may be of a type which measures a BP value of a dorsal portion of foot.

In addition, in each of the second to sixth embodiments shown in FIGS. 7 to 21, the two inflatable cuffs (i.e., pressing bands) 122, 124 are wound around each inferior limb of the patient 112. However, one or more additional cuffs may be wound around a dorsal portion and/or a calf portion of the inferior limb so as to promote the flow of blood in the veins of the inferior limb.

In the fourth embodiment shown in FIG. 16, the photoelectric-pulse-wave sensor 202, 204 is worn on the big toe of the foot. However, the photoelectric-pulse-wave sensor may be worn on a calf or a dorsal portion of the foot.

In the second embodiment shown in FIG. 8, the BP determining means 170, 172, 174 is designed to determine a BP value according to so-called oscillometric method. However, the BP determining means may be designed to determine a BP value according to so-called Korotkoff-sound method in which a cuff pressure at the time when the first one of Korotkoff sounds is detected is determined as a systolic BP value and a cuff pressure at the time when the last Korotkoff sound is detected is determined as a diastolic BP value.

In the third embodiment shown in FIG. 14, the right or left ankle cuff 124R, 124L, the pressure control device 154, 156, and the ankle-cuff-pressure control means 192 cooperate with one another to provide the inferior-limb-pulse-wave detecting device; and in the fourth embodiment shown in FIG. 17, the photoelectric-pulse-wave sensor 202, 204 provides the inferior-limb-pulse-wave detecting device. However, since an impedance between two different portions located on opposite sides of cnemial veins decreases when phlebostasis occurs to the veins, the inferior-limb-pulse-wave detecting device may be an impedance-pulse-wave detecting device which includes two electrodes which are worn on the two different portions and detects, through the two electrodes, an impedance pulse wave representing the change of impedance between the two different portions. In this case, one of the two electrodes may be worn on a proximal side of the calf and the other electrode may be worn on a further distal side of the ankle cuff 124 worn on a distal side of the calf.

It is to be understood that the present invention may be embodied with other changes, improvements, and modifications that may occur to a person skilled in the art without departing from the spirit and scope of the invention defined in the appended claims.

What is claimed is:

1. An apparatus for preventing a venous thromboembolism of a living subject, comprising:
an inflatable cuff which is adapted to be wound around a body portion of the subject and applies a pressing pressure to the body portion so as to press the body portion and thereby prevent the venous thromboembolism;
a blood-pressure-relating-information obtaining device which obtains blood-pressure-relating information which changes corresponding to blood pressure of the subject;
a pressing-pressure determining means for determining the pressing pressure of the inflatable cuff, based on the blood-pressure-relating information obtained by the blood-pressure-relating-information obtaining device, according to a prescribed relationship between pressing pressure and blood-pressure-relating information, in which the pressing pressure increases as the blood pressure corresponding to the blood-pressure-relating information increases; and
a pressing device which operates the inflatable cuff to apply the pressing pressure determined by the pressing-pressure determining means, to the body portion of the subject so as to press the body portion and thereby prevent the venous thromboembolism.

2. An apparatus according to claim 1, further comprising a pressurized-fluid supplying device which supplies a pressurized fluid to the inflatable cuff so as to inflate the cuff, wherein the pressing device comprises a fluid-pressure control device which controls a pressure of the pressurized fluid present in the inflatable cuff so that the cuff applies the determined pressing pressure to the body portion of the subject.

3. An apparatus according to claim 2, wherein the fluid-pressure control device comprises a memory which stores the prescribed relationship in which the pressing pressure monotonously changes as the blood-pressure-relating information changes.

4. An apparatus according to claim 3, wherein the memory stores the prescribed relationship in which the pressing pressure monotonously increases as the blood-pressure-relating information increases.

5. An apparatus according to claim 3, wherein the memory stores the prescribed relationship in which the pressing pressure monotonously increases as the blood-pressure-relating information decreases.

6. An apparatus according to claim 1, wherein the blood-pressure-relating-information obtaining device comprises a blood-pressure measuring device which measures, as the blood-pressure-relating information, the blood pressure of the subject.

7. An apparatus for preventing a venous thromboembolism of a living subject, comprising:
at least two pressing bands which are adapted to be wound around a distal-side portion and a proximal-side portion of an inferior limb of the subject that are located on a distal side and a proximal side of a calf of the inferior limb, respectively, and which apply respective changeable pressing forces to the distal-side portion and the proximal-side portion, such that a distal-side one of the pressing bands earlier starts applying a corresponding one of the changeable pressing forces to the distal-side portion than the other, proximal-side pressing band starts applying the other changeable pressing force to the proximal-side portion, so as to promote flow of blood in veins of the inferior limb and thereby prevent the venous thromboembolism;
a phlebostasis-relating-information obtaining device which obtains, from at least physical information obtained from a distal-side portion of the inferior limb that is located on a distal side of a knee of the subject, phlebostasis-relating information which changes in relation with phlebostasis of the veins of the inferior limb;
a phlebostasis judging means for judging that the veins of the inferior limb have phlebostasis, when the phlebostasis-relating information obtained by the phlebostasis-relating-information obtaining device does not fall within a reference range; and
a blood-flow promoting means for operating, when the phlebostasis judging means judges that the veins of the inferior limb have phlebostasis, the distal-side and proximal-side pressing bands to apply the respective changeable pressing forces to the distal-side and proximal-side portions of the inferior limb, such that the distal-side pressing band earlier starts applying the one changeable pressing force to the distal-side portion of the inferior limb than the proximal-side pressing band starts applying the other changeable pressing force to the proximal-side portion of the inferior limb, so as to promote the flow of blood in the veins of the inferior limb and thereby prevent the venous thromboembolism.

8. An apparatus according to claim 7, further comprising:
a superior-limb-blood-pressure measuring device which iteratively measures a superior-limb blood pressure of a superior limb of the subject; and
an inferior-limb-blood-pressure measuring device which iteratively measures, as the physical information, an inferior-limb blood pressure of the distal-side portion of the inferior limb that is located on the distal side of the knee,
wherein the phlebostasis-relating-information obtaining device iteratively obtains a piece of phlebostasis-relating information based on each of the superior-limb blood pressure values iteratively measured by the superior-limb-blood-pressure measuring device and each of the inferior-limb blood pressure values iteratively measured by the inferior-limb-blood-pressure measuring device.

9. An apparatus according to claim 8, wherein the inferior-limb-blood-pressure measuring device comprises an inflatable cuff which is adapted to be wound around the distal-side portion of the inferior limb that is located on the distal side of the knee, so as to measure the inferior-limb blood pressure, and wherein the distal-side pressing band comprises the inflatable cuff.

10. An apparatus according to claim 7, wherein further comprising:
an inferior-limb-pulse-wave detecting device which detects, as the physical information, an inferior-limb pulse wave from the distal-side portion of the inferior limb that is located on the distal side of the knee; and
a pulse-wave-amplitude determining means for determining an amplitude of each of heartbeat-synchronous pulses of the inferior-limb pulse wave detected by the inferior-limb-pulse-wave detecting device,
wherein the phlebostasis-relating-information obtaining device iteratively obtains a piece of phlebostasis-relating information based on the determined amplitude of said each of the heartbeat-synchronous pulses of the inferior-limb pulse wave.

11. An apparatus according to claim 8, wherein the inferior-limb-pulse-wave detecting device comprises an inflatable cuff which is adapted to be wound around the distal-side portion of the inferior limb that is located on the distal side of the knee, so as to detect the inferior-limb pulse wave, and wherein the distal-side pressing band comprises the inflatable cuff.

12. An apparatus according to claim 7, wherein further comprising:
an inferior-limb-pulse-wave detecting device which detects, as the physical information, an inferior-limb pulse wave from the distal-side portion of the inferior limb that is located on the distal side of the knee; and
a sharpness-degree determining means for determining a degree of sharpness of each of heartbeat-synchronous pulses of the inferior-limb pulse wave detected by the inferior-limb-pulse-wave detecting device,
wherein the phlebostasis-relating-information obtaining device iteratively obtains a piece of phlebostasis-relating information based on the determined degree of sharpness of said each of the heartbeat-synchronous pulses of the inferior-limb pulse wave.

13. An apparatus according to claim 12, wherein the inferior-limb-pulse-wave detecting device comprises a photoelectric-pulse-wave sensor which detects, as the inferior-limb pulse wave, a photoelectric pulse wave from the distal-side portion of the inferior limb that is located on the distal side of the knee.

14. An apparatus according to claim 7, further comprising a weight measuring device which supports an under-knee portion of the inferior limb of the living subject who is taking a face-up position, and which iteratively measures a weight of the under-knee portion, wherein the phlebostasis-relating-information obtaining device iteratively obtains a piece of phlebostasis-relating information based on each of the iteratively measured weights of the under-knee portion.

15. An apparatus according to claim 7, further comprising a circumferential-length measuring device which iteratively measures a circumferential length of a portion of the inferior limb that is located between a knee thereof and an ankle thereof, wherein the phlebostasis-relating-information obtaining device iteratively obtains a piece of phlebostasis-relating information based on each of the iteratively measured circumferential lengths of the under-knee portion.
